SEQUENCE LISTING (82 sequences)

SEQ ID NO: 1 - protein, 228 aa, Homo sapiens
MSVPGPYQAATGPSSAPSAPPSYEETVAVNSYYPTPPAPMPGPTTGLVTGPDGKGMNPPSYYTQPAPIPNNNPITVQTVYVQHPITFLDRPIQMCCPSCNKMIVSQLSYNAGALTWLSCGSLCLLGVHSGLLLHPLLRGCPAGRGPLLSQLQSSPGHLQAFVGLSQTQREPGAAGSPFHLSSSFTPGGGSALVVSPLQGAHLHVFFWGEYVAKLTNLQTPEIAAWSRA

SEQ ID NO: 2 - protein, 404 aa, Homo sapiens
MARRNPSPPLQRLRDWGHHHCPCHPGPGWLSTDGEHPAILCQRPVHSLTGGPNPGSCSAQKSLSQEAQGGGFPEPLQAWAGTGCSDSFLSPTSSLLPAALLSLPVCHPDVTPYFIAVLLLLPHDPCPPFPPPFLPNPRALHGCLFLLTTAEQMGKDGRGYVPATIKMTVERGVWWYGQWVGQGLVVLICRKEGWHQPLVSHMYLLPSSRDQPLPTPELQMPTMVPSYDLGMAPDSSMSMQLGPDMVPQVYPPHSHSIPPYQGLSPEESVNVLSAFQEPHLQMPPSLGQMSLSFDQPHPQGLLPCQPQEHAVSSPDPLLCSDVTTVEDSCLSQPVTAFPQGTWIGEDIFPPLLPPTEQDLTKLLLEGQGESGGGSLGAQPLLQPSHYGQSGISMSHMDLRANPSW

SEQ ID NO: 3 - protein, 12 aa, Homo sapiens
MSVPGPYQAATG

SEQ ID NO: 4 - DNA, 2190 bp, Homo sapiens
  CDS (194)..(1405)
caatgacaacagcctcagtatggaggccttccagcaccgttctgtgtcctatcctgctgggccgtggcttcaccttttggcagtcctttgatggtgtcctggacctcaccaaacgctgtctccggagctactggtctgaccggctgatcattggcttcatcagcaaacagtacgttactagccttcttctcaaatggcccgacggaacccttctcctccgcttcagcgactcagagattgggggcatcaccattgcccatgtcatccggggccaggatggctctccacagatggagaacatccagccattctctgccaaagacctgtccattcgctcactgggggaccgaatccgggatcttgctcagctcaaaaatctctatcccaagaagcccaaggaggaggctttccggagccactacaagcgtgggctggaactggctgctctgattccttcctgtcacccacttcctccctgctccccgctgccctcctctccctgcccgtgtgtcatcctgatgtcactccctatttcatagctgtgcttctcttacttccccatgatccatgcccaccttttccacctcccttcctccctaaccccagagcactccatggctgtcttttccttctcacaacagctgaacagatgggtaaggatggcaggggttatgtcccagctaccatcaagatgaccgtggaaaggggagtgtggtggtatggacagtgggtaggtcaggggctggtagtgcttatctgcaggaaggaggggtggcatcaacccttggtcagtcacatgtacctccttccctcctccagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgtcctttgaccagcctcacccccagggcctgctgccgtgccagcctcaggagcatgctgtgtccagccctgaccccctgctctgctcagatgtgaccacggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtgatcccagctggagggagaacccaaagagacagctcttctactacccccacagacctgctctggacacttgctcatgccctgccaagcagcagatggggagggtgccctcctatccccacctactcctgggtcaggaggaaaagactaacaggagaatgcacagtgggtggagccaatccactccttcctttctatcattcccctgcccacctccttccagcactgactggaagggaagttcaggctctgagacacgccccaacatgcctgcacctgcagcgcgcacacgcacgcacacacacatacagagctctctgagggtgatggggctgagcaggaggggggctgggtaagagcacaggttagggcatggaaggcttctccgcccattctgacccagggcctaggacggataggcaggaacatacagacacatttacactagaggccagggatagaggatattgggtctcagccctaggggaatgggaagcagctgaagggaccctgggtgggagcataggaggagtctggacatgtggttactagtacaggttttgccctgattaaaaaatctcccaaagccccaaattcctgttagccaggtggaggcttctgatacgtgtatgagactatgcaaaagtacaagggctgagattcttcgtgtatagctgtgtgacgtgtatgtacctaggatatgttaaatatatagctggcaccttagttgcatgaccaaaaaaaaaaaaaaaaaaaaaaaaaaaaaaaaaaaaaaaaaaaaaaaaa

SEQ ID NO: 5 - DNA, 21 bp, Artificial Sequence
  Description of Artificial Sequence Synthetic primer
tgccatgagctctccgccgcc

SEQ ID NO: 6 - DNA, 21 bp, Artificial Sequence
  Description of Artificial Sequence Synthetic primer
tcacagaggcgtctccttgga

SEQ ID NO: 7 - DNA, 21 bp, Artificial Sequence
  Description of Artificial Sequence Synthetic primer
atggctcggatgaaccgcccg

SEQ ID NO: 8 - DNA, 21 bp, Artificial Sequence
  Description of Artificial Sequence Synthetic primer
ctacataacgcagcaccgggt

SEQ ID NO: 9 - DNA, 22 bp, Artificial Sequence
  Description of Artificial Sequence Synthetic primer
atggcccgacggaacccttctc

SEQ ID NO: 10 - DNA, 21 bp, Artificial Sequence
  Description of Artificial Sequence Synthetic primer
gagggcagcggggagcaggga

SEQ ID NO: 11 - DNA, 22 bp, Artificial Sequence
  Description of Artificial Sequence Synthetic primer
atggcccgacggaacccttctc

SEQ ID NO: 12 - DNA, 21 bp, Artificial Sequence
  Description of Artificial Sequence Synthetic primer
actgaccaagggttgatgcca

SEQ ID NO: 13 - DNA, 22 bp, Artificial Sequence
  Description of Artificial Sequence Synthetic primer
atggcccgacggaacccttctc

SEQ ID NO: 14 - DNA, 21 bp, Artificial Sequence
  Description of Artificial Sequence Synthetic primer
gccctgggggtgaggctggtc

SEQ ID NO: 15 - DNA, 22 bp, Artificial Sequence
  Description of Artificial Sequence Synthetic primer
atggcccgacggaacccttctc

SEQ ID NO: 16 - DNA, 21 bp, Artificial Sequence
  Description of Artificial Sequence Synthetic primer
tcaccaactggggttggccct

SEQ ID NO: 17 - DNA, 26 bp, Artificial Sequence
  Description of Artificial Sequence Synthetic primer
cgggatccatgtcggttccaggacct

SEQ ID NO: 18 - DNA, 26 bp, Artificial Sequence
  Description of Artificial Sequence Synthetic primer
cggaattcggtaattggattgttatt

SEQ ID NO: 19 - DNA, 26 bp, Artificial Sequence
  Description of Artificial Sequence Synthetic primer
cgggatccatgtcggttccaggacct

SEQ ID NO: 20 - DNA, 26 bp, Artificial Sequence
  Description of Artificial Sequence Synthetic primer
cggaattccagttgggacagtaatgg

SEQ ID NO: 21 - DNA, 26 bp, Artificial Sequence
  Description of Artificial Sequence Synthetic primer
cgggatccgtgcagacggtctacgtg

SEQ ID NO: 22 - DNA, 26 bp, Artificial Sequence
  Description of Artificial Sequence Synthetic primer
cggaattccagttgggacagtaatgg

SEQ ID NO: 23 - DNA, 26 bp, Artificial Sequence
  Description of Artificial Sequence Synthetic primer
cgggatccatgtcggttccaggacct

SEQ ID NO: 24 - DNA, 26 bp, Artificial Sequence
  Description of Artificial Sequence Synthetic primer
cgggatcctcagggtctcagggaggc

SEQ ID NO: 25 - DNA, 26 bp, Artificial Sequence
  Description of Artificial Sequence Synthetic primer
cgggatccgtgcagacggtctacgtg

SEQ ID NO: 26 - DNA, 26 bp, Artificial Sequence
  Description of Artificial Sequence Synthetic primer
cgggatcctcagggtctcagggaggc

SEQ ID NO: 27 - DNA, 26 bp, Artificial Sequence
  Description of Artificial Sequence Synthetic primer
cgggatcccagagctctcctgggcac

SEQ ID NO: 28 - DNA, 26 bp, Artificial Sequence
  Description of Artificial Sequence Synthetic primer
cgggatcctcagggtctcagggaggc

SEQ ID NO: 29 - DNA, 24 bp, Artificial Sequence
  Description of Artificial Sequence Synthetic primer
aatgtttctagattctcctttagc

SEQ ID NO: 30 - DNA, 24 bp, Artificial Sequence
  Description of Artificial Sequence Synthetic primer
cctctcggttcctctggctgctgt

SEQ ID NO: 31 - DNA, 24 bp, Artificial Sequence
  Description of Artificial Sequence Synthetic primer
aatgtttctagattctcctttagc

SEQ ID NO: 32 - DNA, 34 bp, Artificial Sequence
  Description of Artificial Sequence Synthetic primer
gatggggtgccattaagcccagactgaccacaga

SEQ ID NO: 33 - DNA, 27 bp, Artificial Sequence
  Description of Artificial Sequence Synthetic primer
gcaccccatccatttgctcatttggtc

SEQ ID NO: 34 - DNA, 24 bp, Artificial Sequence
  Description of Artificial Sequence Synthetic primer
cctctcggttcctctggctgctgt

SEQ ID NO: 35 - DNA, 24 bp, Artificial Sequence
  Description of Artificial Sequence Synthetic primer
aatgtttctagattctcctttagc

SEQ ID NO: 36 - DNA, 24 bp, Artificial Sequence
  Description of Artificial Sequence Synthetic primer
cctctcggttcctctggctgctgt

SEQ ID NO: 37 - DNA, 24 bp, Artificial Sequence
  Description of Artificial Sequence Synthetic primer
aatgtttctagattctcctttagc

SEQ ID NO: 38 - DNA, 31 bp, Artificial Sequence
  Description of Artificial Sequence Synthetic primer
agagggcggagtcaagcaggaggagggatct

SEQ ID NO: 39 - DNA, 32 bp, Artificial Sequence
  Description of Artificial Sequence Synthetic primer
ctccgccctcttctgcccgctttcaataagag

SEQ ID NO: 40 - DNA, 24 bp, Artificial Sequence
  Description of Artificial Sequence Synthetic primer
cctctcggttcctctggctgctgt

SEQ ID NO: 41 - DNA, 24 bp, Artificial Sequence
  Description of Artificial Sequence Synthetic primer
aatgtttctagattctcctttagc

SEQ ID NO: 42 - DNA, 24 bp, Artificial Sequence
  Description of Artificial Sequence Synthetic primer
cctctcggttcctctggctgctgt

SEQ ID NO: 43 - DNA, 24 bp, Artificial Sequence
  Description of Artificial Sequence Synthetic primer
aatgtttctagattctcctttagc

SEQ ID NO: 44 - DNA, 34 bp, Artificial Sequence; identical to SEQ ID NO: 32
  Description of Artificial Sequence Synthetic primer

SEQ ID NO: 45 - DNA, 27 bp, Artificial Sequence
  Description of Artificial Sequence Synthetic primer
gcaccccatccatttgctcatttggtc

SEQ ID NO: 46 - DNA, 24 bp, Artificial Sequence
  Description of Artificial Sequence Synthetic primer
cctctcggttcctctggctgctgt

SEQ ID NO: 47 - DNA, 27 bp, Artificial Sequence
  Description of Artificial Sequence Synthetic primer
gcaccccatccatttgctcatttggtc

SEQ ID NO: 48 - DNA, 24 bp, Artificial Sequence
  Description of Artificial Sequence Synthetic primer
cctctcggttcctctggctgctgt

SEQ ID NO: 49 - DNA, 27 bp, Artificial Sequence
  Description of Artificial Sequence Synthetic primer
gcaccccatccatttgctcatttggtc

SEQ ID NO: 50 - DNA, 24 bp, Artificial Sequence
  Description of Artificial Sequence Synthetic primer
cctctcggttcctctggctgctgt

SEQ ID NO: 51 - DNA, 27 bp, Artificial Sequence
  Description of Artificial Sequence Synthetic primer
gcaccccatccatttgctcatttggtc

SEQ ID NO: 52 - DNA, 24 bp, Artificial Sequence
  Description of Artificial Sequence Synthetic primer
cctctcggttcctctggctgctgt

SEQ ID NO: 53 - protein, 11 aa, Artificial Sequence
  Description of Artificial Sequence Synthetic peptide
YPYDVPDYASL

SEQ ID NO: 54 - DNA, 22 bp, Artificial Sequence
  Description of Artificial Sequence Synthetic primer
atctggggttgggggggcagca

SEQ ID NO: 55 - DNA, 21 bp, Artificial Sequence
  Description of Artificial Sequence Synthetic primer
catggtttcggaggcccgacc

SEQ ID NO: 56 - DNA, 22 bp, Artificial Sequence
  Description of Artificial Sequence Synthetic primer
atctggggttgggggggcagca

SEQ ID NO: 57 - DNA, 21 bp, Artificial Sequence
  Description of Artificial Sequence Synthetic primer
agggacacacagatctgttgg

SEQ ID NO: 58 - DNA, 33 bp, Artificial Sequence
  Description of Artificial Sequence Synthetic primer
ctgtgtgtccctcacccgtccctgtccggctct

SEQ ID NO: 59 - DNA, 21 bp, Artificial Sequence
  Description of Artificial Sequence Synthetic primer
catggtttcggaggcccgacc

SEQ ID NO: 60 - DNA, 22 bp, Artificial Sequence
  Description of Artificial Sequence Synthetic primer
atctggggttgggggggcagca

SEQ ID NO: 61 - DNA, 21 bp, Artificial Sequence
  Description of Artificial Sequence Synthetic primer
catggtttcggaggcccgacc

SEQ ID NO: 62 - DNA, 22 bp, Artificial Sequence
  Description of Artificial Sequence Synthetic primer
atctggggttgggggggcagca

SEQ ID NO: 63 - DNA, 21 bp, Artificial Sequence
  Description of Artificial Sequence Synthetic primer
caggaaagtgaggttacgtgc

SEQ ID NO: 64 - DNA, 32 bp, Artificial Sequence
  Description of Artificial Sequence Synthetic primer
tcactttcctgtcctcgccaatgccccgcggg

SEQ ID NO: 65 - DNA, 21 bp, Artificial Sequence
  Description of Artificial Sequence Synthetic primer
catggtttcggaggcccgacc

SEQ ID NO: 66 - DNA, 22 bp, Artificial Sequence
  Description of Artificial Sequence Synthetic primer
atctggggttgggggggcagca

SEQ ID NO: 67 - DNA, 21 bp, Artificial Sequence
  Description of Artificial Sequence Synthetic primer
catggtttcggaggcccgacc

SEQ ID NO: 68 - DNA, 22 bp, Artificial Sequence
  Description of Artificial Sequence Synthetic primer
atctggggttgggggggcagca

SEQ ID NO: 69 - DNA, 21 bp, Artificial Sequence
  Description of Artificial Sequence Synthetic primer
agggacacacagatctgttgg

SEQ ID NO: 70 - DNA, 33 bp, Artificial Sequence
  Description of Artificial Sequence Synthetic primer
ctgtgtgtcccttcctcgccaatgccccgcggg

SEQ ID NO: 71 - DNA, 21 bp, Artificial Sequence
  Description of Artificial Sequence Synthetic primer
catggtttcggaggcccgacc

SEQ ID NO: 72 - DNA, 22 bp, Artificial Sequence
  Description of Artificial Sequence Synthetic primer
atctggggttgggggggcagca

SEQ ID NO: 73 - DNA, 21 bp, Artificial Sequence
  Description of Artificial Sequence Synthetic primer
catggtttcggaggcccgacc

SEQ ID NO: 74 - DNA, 21 bp, Artificial Sequence
  Description of Artificial Sequence Synthetic primer
tctgtgtgtccctctccccac

SEQ ID NO: 75 - DNA, 21 bp, Artificial Sequence
  Description of Artificial Sequence Synthetic primer
catggtttcggaggcccgacc

SEQ ID NO: 76 - DNA, 21 bp, Artificial Sequence
  Description of Artificial Sequence Synthetic primer
ctcctcgccaatgccccgcgg

SEQ ID NO: 77 - DNA, 21 bp, Artificial Sequence
  Description of Artificial Sequence Synthetic primer
catggtttcggaggcccgacc

SEQ ID NO: 78 - DNA, 21 bp, Artificial Sequence
  Description of Artificial Sequence Synthetic primer
ctctttagccagagccggggt

SEQ ID NO: 79 - DNA, 21 bp, Artificial Sequence
  Description of Artificial Sequence Synthetic primer
catggtttcggaggcccgacc

SEQ ID NO: 80 - DNA, 21 bp, Artificial Sequence
  Description of Artificial Sequence Synthetic primer
tgccgctcactttgatgtctg

SEQ ID NO: 81 - DNA, 21 bp, Artificial Sequence
  Description of Artificial Sequence Synthetic primer
catggtttcggaggcccgacc

SEQ ID NO: 82 - protein, 847 aa, Homo sapiens
MSLWGLVSKMPPEKVQRLYVDFPQHLRHLLGDWLESQPWEFLVGSDAFCCNLASALLSDTVQHLQASVGEQGEGSTILQHISTLESIYQRDPLKLVATFRQILQGEKKAVMEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWDNAFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

(19) United States
(12) Patent Application Publication
(10) Pub. No.: US 2009/0062190 A1
(43) Pub. Date: Mar. 5, 2009

(54) Transcriptional Regulation of Cytokines by LITAF and STAT (6)(B)

(76) Inventors: Salomon Amar, Brookline, MA (US); Xiaoren Tang, Winchester, MA (US)

Correspondence Address: Kevin M. Farrell;Pierce Atwood, One New Hampshire Ave., Suite 350, Portsmouth, NH, 03801, US

(21) Appl. No.: 11/884,854

(22) PCT Filed: Feb. 23, 2006
(86) PCT No.: PCT/US06/06576

§ 371 Date: Jul. 31, 2008

Related U.S. Application Data
(60) Provisional application No. 60/655,851, filed on Feb. 24, 2005.

(51) Int. Cl. A61K 38/00; C12N 15/00; C07K 14/00; C12N 5/06; A61P 31/00; A61K 31/7088; A01K 67/027; C40B 30/04

(52) U.S. Cl. 514/12; 435/320.1; 530/350; 435/325; 435/375; 800/13; 800/15; 800/16; 800/18; 506/9; 514/44

(57) ABSTRACT

The present invention relates to novel proteins (LITAF and STAT6(B)) and the nucleotide sequences encoding the same. The present invention also relates to the use of the novel peptides and nucleotide sequences of the present invention, or functional fragments thereof, for the regulation of cytokine expression. The present invention also relates to the use of the novel proteins and nucleotides sequences of the present invention for the regulation of inflammatory responses in mammals including the regulation of angiogenesis and tubulogenesis. Also in this regard, the present invention relates to the generation of null mutant animals deficient in the expression of one or both of the proteins of the present invention.

### GOVERNMENT SUPPORT

This invention was made with Government Support under Contract No. DE 14079 awarded by the National Institute of Health. The Government has certain rights in the invention.

### FIELD OF THE INVENTION

This invention relates to the transcriptional regulation of cytokines by the proteins LITAF and STAT6(B).

### BACKGROUND

Cytokines are critical players in both the innate and adaptive immune systems. They activate complement, trigger phagocytosis, raise body temperature, and activate B and T cells. All of these functions are vital to host defense, but cytokine expression must be carefully regulated because these mediators have both favorable and deleterious effects. For example, overproduction of cytokines is thought to contribute to the development of diseases such as diabetes, atherosclerosis, and rheumatoid arthritis as well as to inappropriate allergic reactions (Eu, et al., Biochemistry, 39:1375-1384, 2000; Rapoport, et al., Cytokine, 30:219-227, 2005). Conversely, insufficient production of cytokines, resulting in immune suppression, can allow mild opportunistic infections to progress, and even to become deadly (Bronke, et al., Immunol Lett, 97:215-224; Cecere, et al., Panminerva Med, 46:171-187, 2004; Nicolle, Lab Invest, 84:1305-1321, 2004). Therefore, understanding the control of cytokine gene expression is crucial to understanding many disease states.

Cytokines modulate an immune cell's coordinated response to infection by triggering signal cascades, such as the interferon-induced JAK-STAT transcriptional pathway (Darnell, et al., Science, 264:1415-1421, 1994) that induce or suppress the expression of target genes. Thus, a specific cytokine profile and its subsequent gene induction are responsible for pro- or anti-inflammatory cellular responses, cell differentiation, and proliferation. Cytokine-mediated responses, specifically those that are activated through JAK-STAT signaling, are known to be regulated by a variety of controls, including suppressor of cytokine signaling (SOCS) proteins (Alexander, et al., Annu Rev Immunol, 22:503-529, 2004), the protein inhibitor of activated STAT family (PIAS), protein tyrosine phosphatases (PTPs), STAT and JAK protein modifications and cross-talk between JAK-STAT pathways (Chen, et al., J Immunol, 172:6744-6750, 2004; Luo, et al., Drug Discov Today, 9:268-275, 2004; Shuai, et al., Nat Rev Immunol, 3:900-911, 2003). Many of these regulators and mechanisms act via negative-feedback loops, providing crucial signaling restraints to cytokine-induced effects at multiple levels of regulation, emphasizing the importance of controlled cytokine signaling.

Regulation of inflammatory cytokines, which are produced predominantly by activated immune cells, is only partially understood. Therefore, what is needed are new compositions and methods of i) deducing the regulation of inflammatory cytokines and ii) modulating the activation of inflammatory cytokines.

### SUMMARY OF THE INVENTION

The present invention relates to novel proteins and their associated gene sequences that are effective in the activation and/or regulation of inflammatory cytokines. In particular, the present invention relates to the novel protein that we named STAT6(B) (Signal transducer and activator of transcription; SEQ ID NO: 2; GenBank No. AY615283) and the nucleotide sequences encoding said novel peptide (SEQ ID NO: 4) as well as compositions and methods comprising said protein (or functional portions thereof) and said nucleotide sequences (and portions encoding functional protein fragments).

The present invention also relates to the interaction of STAT6(B) with the protein named LITAF (LPS-induced TNF-α factor; SEQ ID NO: 1) as well as compositions and methods comprising the nucleotide sequence encoding LITAF (SEQ ID NO: 3) and functional portions of said protein and portions of said nucleotide sequence encoding said function protein portions. In the present invention the term “interaction” refers to the transient or permanent binding of STAT6(B) and LITAF, for example, to each other under physiological conditions or under laboratory test conditions. Such conditions may be, for example, between about pH 5.0 to 8.0, and between about 4° C. to 50° C.

The present invention also relates to the use of the proteins and nucleotide sequences of the present invention in compositions. Such compositions include, for example, expression vectors comprising SEQ ID NO: 3 and/or SEQ ID NO: 4 or portions suitable for the expression of functional peptides or peptide fragments. Functional peptides or functional peptide fragments shall be defined as amino acid sequences capable of performing, to a measurable extent, essentially the same function as the full protein.

The present invention also relates to the use of the peptides and nucleotide sequences of the present invention, or functional fragments thereof for the regulation of cytokine expression in organisms, tissues, cells, cell-free systems and diagnostic or assay systems and protocols. In this regard, the peptides of the present invention or nucleic acid sequences encoding said peptides of the present invention might be transfected into cells, tissues or organisms for the purpose of modulating cytokine activity. Also in this regard, the present invention relates to the regulation of angiogenesis and tubulogenesis by the proteins or functional protein fragments of the present invention via the regulation of cytokine expression as well as pharmaceutical compositions comprising the proteins or functional protein fragments of the present invention.

The present invention also relates to the use of the novel proteins and nucleotides sequences of the present invention for the regulation of inflammatory responses in mammals. Also in this regard, the present invention relates to the generation of null mutant animals wherein said animals do not express functional copies of either LITAF, STAT6(B) or both LITAF and STAT6(B).

### BRIEF DESCRIPTION OF THE DRAWINGS

FIG. 1 shows (a) the nucleotide and amino acid sequences of STAT6(B); (b) differences between the amino acid sequences of STAT(6)A [NM_{—}003153; GenBank] and STAT6(B) and; (c) Northern blots of STAT(6)A and STAT6(B) RNA.

FIG. 2 shows Western blotting of STAT6(B) and LITAF after induction by LPS.

FIG. 3 shows a dose dependent response by STAT6(B) to LPS.

FIG. 4 shows an EMSA showing that STAT6(B) possesses a binding domain for LITAF.

FIG. 5 shows the translocation of the LITAF-STAT6(B) complex in THP-1 cells detected by Western blotting: (a) LITAF and (b) STAT6(B).

FIG. 6 (a-d) shows the regulation of various cytokines by STAT6(B) and/or LITAF.

FIG. 7 (a-d) shows the effect of silencing LITAF expression with LITAF-specific shRNA.

FIG. 8 (a-b) shows the genotyping of LITAF−/− homozygous and wild type mice by PCR and Western blot.

FIG. 9 (a-c) shows the production of TNF-α by LITAF+/+ and LITAF-−/− macrophages exposed to LPS and analyzed by ELISA.

FIG. 10 shows that LITAF−/− mice are immune to lethal doses of LPS compared to LITAF+/+mice.

FIG. 11 (a) shows a diagram of the transient transfection of the identified DNA into U₂OS cells; (b-c) show Western blots of transfected cells for LITAF, STAT6(B) and VEGF expression and; (d) shows schematic diagrams of the pcVEGF and pEVEGF promoter regions.

FIG. 12 shows the results of an angiogenesis/tubulogenesis assay of STAT6(B) induced VEGF.

FIG. 13 shows the effects of kinase on the translocation of STAT6(B) and LITAF in mouse macrophages after LPS plus kinase specific inhibitor treatment: (a) shows the various kinase inhibitors used; (b) shows a Western blot of TNF-α expression after treatment.

FIG. 14 shows a diagram of the transient transfection of DNAs for p38MAPK, PTP STAT6(B) and LITAF.

FIG. 15 shows a human cytokine antibody array and Western blot of DNA-transfected THP-1 cells: (a) shows the density value of each test sample normalized to β-galactosidase: (b) shows a Western blot validation of the cytokine array data.

FIG. 16 shows a luciferase assay of promoter activity of MCP-1 and TNF-α in response to transient transfection of STAT6(B) alone, LITAF alone of both.

FIG. 17 shows a diagram of “CTCCC” on various cytokine promoters.

FIG. 18 shows a diagram of MCP-1 promoter constructs.

FIG. 19 shows a diagram of VEGF promoter constructs.

FIG. 20 shows a diagram of the generation of STAT6(B) knock-out mice.

### DESCRIPTION OF THE INVENTION

The present invention relates to compositions and methods for the use of such compositions in modulating cytokine expression in a cell. The present invention is based in part on the identification of a novel gene that belongs to the Signal Transducers and Activators of Transcription (STAT) family. This novel gene, termed STAT6(B), was found to modulate the expression of various cytokines when introduced into a cell with or without concurrent introduction of LITAF. Interestingly, introduction of LITAF without concurrent introduction of STAT6(B) was also found to modulate the expression of certain cytokines. Prior to the present invention, the effect of STAT6(B) or STAT6(B) and LITAF on cytokine expression was unknown. Additionally, the invention provides the first use of LITAF for modulating the expression of cytokines other than TNF-α.

Although the present invention is not limited to any particular theory, it is believed that STAT6(B) and LITAF establish an equilibrium in the cell wherein a change in the equilibrium results in the activation or inhibition of, for example, various cytokines produced by the cell. Methods of changing this equilibrium include, for example, increasing or decreasing either of STAT6(B) or LITAF by direct injection or endocytosis of the protein into the cell, expression of exogenous genes encoding STAT6(B) or LITAF, by limiting the effect of endogenous expression of STAT6(B) or LITAF through expression of exogenous non-functional STAT6(B) or LITAF (wherein the balance established between STAT6(B) and LITAF would be affected since functional versions one of the proteins would be out competed by the non-functional versions), administration of compounds known or suspected of inhibiting or promoting STAT6(B) and LITAF interaction (either by blocking binding sites on either of the molecules or sequestering one or both of the molecules), etc.

Methods of introducing proteins into a cell or cells are well known in the art and are provided in detail in, for example, Sambrook, J., Fritsch, E. F. and Maniatis, T., Molecular Cloning: A Laboratory Manual. Cold Spring Harbor Laboratory Press, NY, Vol. 1, 2, 3 (1989), which is incorporated herein by reference. In brief, proteins may be introduced into cells in two general ways. The first is with the transfection of nucleic acids expressing the desired protein. Once inside the cell the protein is produced by the cell's own transcription and translation components. The second is with the transfection of the actual protein.

Transfection of nucleic acids is well known in the art. The nucleic acid sequence (for example, the nucleic acid sequences of the present invention or portions that encode functional fragments of the encoded proteins) encoding the desired protein is operably inserted into an expression vector suitable for the transcription and translation of the nucleic acid sequence into the desired protein. A large number of expression vectors are commercially available (e.g., pCAT, Promega, Madison, Wis.; pBlueScript and PCMV, Stratagene, La Jolla, Calif.). The splicing of a specific nucleotide sequence into an expression vector is also well know in the art as is referenced above in Sambrook, et al. Suitable transfection methods include DEAE-dextran calcium phosphate precipitation, Lipofectamine™ (Invitrogen, Carlsbad, Calif.), Profectin™ (Promega, Madison, Wis.) and other liposome methods, direct microinjection, electroporation and bioloastic particle delivery, for example. Any primary cell or cell line may be used for the present invention.

Transfection of the actual proteins of the present invention (or functional fragments thereof) may take place by, for example, transport (active or passive) or by microinjection. Active transport is a process whereby cells absorb material from the outside the cell by engulfing it with the cell membrane. Passive transport may also take place via the passage of peptide fragments, for example, into the cell through pores. Cells frequently transport particles and, especially, proteins and protein fragments into the cell's cytoplasm. Transport may be specific via, for example, specific receptors or it may be more general. With general forms of active transport the cell engulfs constituents from the extracellular milieu. This is often referred to as pinocytosis. Pinocytosis (literally, cell-drinking) is the invagination of the cell membrane to form a pocket filled with extracellular fluid (and molecules within it). The pocket then pinches off to form a vesicle, and the vesicle ruptures to release its contents into the cytoplasm.

In addition to the techniques given directly above, transfection of the proteins of the present invention may also be executed by techniques used for the transfection of nucleic acids into cells, as given above. In brief, suitable transfection methods include DEAE-dextran, calcium phosphate precipitation, Lipofectamine™ (Invitrogen, Carlsbad, Calif.), Profectin™ (Promega, Madison, Wis.) and other liposome methods, direct microinjection, electroporation and bioloastic particle delivery, for example. Any primary cell or cell line may be used for the present invention.

In one embodiment, the present invention contemplates the identification of active fragments of either STAT6(B) and LITAF. These fragments could be identified by randomly or selectively cleaving the full proteins and testing the effect of the fragment of cytokine expression. Alternatively, the protein fragments could be produced by transfection into cells of nucleic acids encoding any of the specific fragments. One skilled into the art would be able to determine which fragments may be suitable for testing for activity based on the sequence or configuration of the fragment. For example, active sites of protein typically form a three dimensional cup or bowl shape.

As explained in further detail below, the introduction of STAT6(B) and/or LITAF into a cell, tissue or organism is useful for the regulation of various cytokines and the physiological processes the regulate such as, for example, angiogenesis and tubulogenesis.

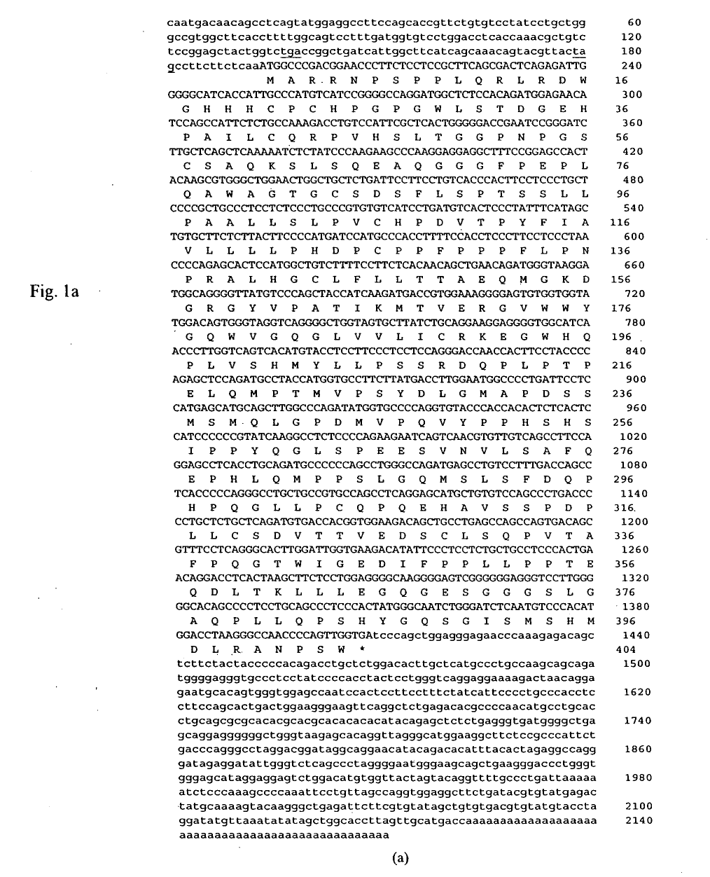

A method for modulating cytokine expression may include, for example, introducing into a cytokine-responsive cell a composition comprising STAT6(B) (SEQ ID NO 2: FIG. 1(a), amino acid sequence) effective to modulate cytokine expression in the cell. The cytokine to be modulated may be IL-1α, IL-10, GRO, VEGF and/or RANTES. Introduction of STAT6(B) into the cell increases expression of IL-1α, IL-10, GRO, VEGF and RANTES and, as such, this method may be used to effect any of the cellular processes resulting from the same.

This method for modulating cytokine expression may further include introducing into the cell to which STAT6(B) was introduced a composition comprising LITAF. The LITAF is to be introduced in a manner effective to form a complex of the introduced LITAF with the introduced STAT6(B) in the cell. There is no requirement as to the order of STAT6(B) and LITAF introduction and, resultingly, LITAF may be introduced into the cell prior to, subsequent to, or concurrent with the introduction of STAT6(B). The cytokine to be modulated may be TNF-α, IL-1α, IL-10, GRO, RANTES, IFN-γ, VEGF, MCP-1 and/or MCP-2. Introduction of STAT6(B) and LITAF into the cell increases expression of TNF-α, IL-1α, IL-10, GRO, RANTES, IFN-γ, VEGF, MCP-1 and MCP-2 and, as such, this method may be used to effect any of the cellular processes resulting from the same.

Alternatively, a method for modulating cytokine expression may include introducing into a cytokine-responsive cell a composition comprising LITAF, without the introduction of exogenous STAT6(B) as described above. In this method, LITAF is to be introduced into the cell in an amount effective to modulate cytokine expression. The cytokine to be modulated may be TNF-α VEGF and/or IL-1β. Introduction of LITAF into the cell increases expression of TNF-α and IL-1β and decreases expression of VEGF and, as such, this method may be used to effect any of the cellular processes resulting from the same.

One of skill in the art will recognize that a biologically active fragment of STAT6(B) and/or LITAF may be used in lieu of the full-length sequence or sequences in the context of the present invention. A “biologically active fragment” is intended to encompass any mimetic, truncation, deletion and/or substitution of full-length STAT6(B) and/or LITAF with the ability to modulate cytokine expression in the methods of the present invention. A biologically active fragment may further be a protein, polypeptide or peptide. As defined in this invention, the terms “protein,” “peptide” or “polypeptide” are interchangeable and refer to a sequence of two of more amino acids with or without additional modifications such as, but not limited to, glycosylation.

One of skill in the art will recognize that STAT6(B) and LITAF or biologically active fragment(s) thereof may be introduced into a cell by various means in the methods of the present invention. A cell may be contacted directly with STAT6(B) and/or LITAF or the biologically active fragment(s) thereof under conditions for cellular uptake. Such conditions include but are not limited to injection and calcium chloride mediated uptake, electroporation, microinjection, etc. Alternatively, a cytokine-responsive cell may express exogenous STAT6(B) and/or LITAF from an introduced exogenous construct harboring an expressible cDNA construct or constructs. As with the proteins, themselves, constructs harboring an expressible nucleotide sequences suitable for the expression of STAT6(B) and/or LITAF or an active fragment thereof, may be introduced into the cell by, for example, calcium chloride mediated uptake, electroporation, micro injection, etc. If LITAF and STAT6(B) are to be introduced concurrently, a single construct harboring both cDNAs may be employed or multiple constructs harboring each cDNA may be used. Such constructs harboring both cDNAs may, for example, transcribe both proteins under one promoter or may express each protein under a separate promoter. Furthermore, in organisms such as animals or individuals, the construct may be delivered by methods of gene therapy, which are known in the art. STAT6(B) and/or LITAF may further be introduced indirectly by increasing the expression of endogenous STAT6(B) and/or LITAF genes as discussed infra. Alternatively, STAT6(B) and/or LITAF RNAs may be delivered to cells by injection (i.e., microinjection) or other delivery means already known in the art.

In the methods of the present invention, a composition comprising STAT6(B) and/or LITAF or biologically active fragment(s) thereof may be administered to an animal or individual in a physiologically acceptable carrier in a therapeutically effective amount. Said compound or compounds may be administered alone or in combination with other therapies and may be delivered intravenously, subcutaneously or orally to an animal. Administration may be systemic although local administration may be preferable.

It is an object of the present invention to employ the methods disclosed herein for modulating cellular responses to cytokine expression. The methods of the present invention may be used to study and/or treat diseases associated with aberrant cytokine signaling. It is known in the art that cytokine signaling is involved in pro-inflammatory and anti-inflammatory responses to pathogens and in cellular proliferation and differentiation in a variety of cells. Thus, methods disclosed herein for modulating cytokine signaling may be used to alter these and other cytokine-dependent processes in normal and/or abnormal cells. As an example, the cytokine VEGF is known to stimulate proper wound healing and vascularization (e.g., angiogenesis) in adult tissues. An increase in VEGF expression as mediated by STAT6(B) in a method of the invention would be useful for stimulating such processes. Vascularization has been implicated in tumor growth and, thus, a decrease in VEGF expression as mediated by the STAT6(B)-LITAF complex in a method of the present invention may be desirable for slowing tumor growth and treating cancer.

Angiogenesis is the physiological process involving the growth of new blood vessels from pre-existing vessels. Angiogenesis is a normal process in growth and development, as well as in wound healing. However, this is also a fundamental step in the transition of tumors from a dormant state to a malignant state and, therefore, the modulation and regulation of angiogenesis maybe critical in the treatment of, for example, wounds and cancers. Modulation of cytokine signaling via the compositions and methods of the present invention may also be used to increase the immune response of an animal or individual to an antigen, or to treat diseases or repair of damage caused by such as diabetes or inflammatory diseases.

Also provided herein are compositions and methods for the treatment of non-healing ulcers, for example, in the context of diabetes. Diabetes typically may cause such ulcers (e.g., tissue damage of the kidney) or be the cause for wounding (e.g., amputation of limbs as a result of disease progression). The compositions and methods of the present invention are ideally suited for the treatment of such conditions via the promotion of angiogenesis.

Also provided herein are compositions for use in the methods disclosed herein. It is an object of the invention to provide an expression vector comprising a nucleic acid sequence which encodes STAT6(B) [SEQ ID NO 2] or a biologically active fragment thereof. The expression vector may include a nucleic acid sequence comprising nucleotide sequence SEQ ID NO: 4 (FIG. 1(a). It is also an object of the invention to provide for an isolated protein comprising the amino acid sequence of STAT6(B) [SEQ ID NO 2], or a biologically active fragment thereof. The isolated protein may be complexed with LITAF (see, U.S. Pat. No. 6,566,501, which is herein incorporated by reference) or a biologically active fragment thereof. A cell that exogenously expresses STAT6(B) [SEQ ID NO: 2] and/or LITAF or biologically active fragment(s) thereof, is also within the scope of the present invention.

The present invention also provides for animals modified to reduce or eliminate the expression of LITAF, STAT6(B) or both. Such animals are termed “knockout,” “null,” “null mutant” or “−/− homozygous” animals. The procedures for the generation of null animals is well known in the art. For example, knockout animals may be generated by disruption of the endogenous gene sequence through a procedure called homologous recombination wherein the native sequence is disrupted with the integration of a targeting vector. Additionally, knockout animals may be generated by crossing heterozygous+/− parents and screening (via Western blotting or PCR, for example) for homozygous off spring. (See, for example, U.S. Pat. No. 5,286,632 to Jones and U.S. Pat. No. 5,612,205 to Kay). Such procedure may be performed in vivo (see, U.S. Pat. No. 6,326,206 to Bjornvad, for example) or in vitro. In one aspect of the present invention, the generation of LITAF−/− homozygous and/or STAT6(B)−/− homozygous animals is contemplated. Animals deficient in the expression of both functional LITAF and STAT6(B) are termed “double null mutant” animals or “double−/− homozygous” animals. The present invention is not limited to the choice of animal used for the creation of a null animal. Examples of suitable animals are mice, rats and other rodents, cats, dogs, cows and other ungulates, etc. In a preferred embodiment, mice are the preferred animals.

Another aspect of the present invention is the use of said null animals or cells from said null animals for the screening of agents (e.g., small molecule compounds) that may inhibit or modulate STAT6(B) and/or LITAF mediated cytokine expression in the absence of STAT6(B) and/or LITAF expression. In one embodiment, for example, the null mutant animal may be used as a control animal in the screening procedure. In one embodiment, a small molecule compound suspected of modulating TNF-α, IL-1α, IL-10, GRO, RANTES, IFN-γ, VEGF, MCP-1 and/or MCP-2 expression is administered to an animal or cell homozygotic for STAT6(B) and/or LITAF and a “wild type” animal that expresses STAT6(B) and/or LITAF. Then, the expression of TNF-α IL-1α, IL-10, GRO, RANTES, IFN-γ, VEGF, MCP-1 and/or MCP-2 are monitored by, for example, Western blotting or PCR analysis. Those practiced in the art will note that there are other uses for null animals and these uses are considered to be embodiments of the present invention.

The present invention also relates to method for the screening of agents (e.g., small molecule compounds) suspected of modulating STAT6(B) and/or LITAF function. For example, in one embodiment, molecules from a library of small molecules are used to form a reaction mixture comprising a small molecule and a cell expressing STAT6(B) and/or LITAF. Expression of STAT6(B) and/or LITAF is monitored by, for example, Western blotting or PCR to detect changes in said expression as compared to controls. Additionally, one or more of the cytokines modulated by STAT6(B) and/or LITAF may be monitored for changes in expression.

Another aspect of the present invention relates to the screening of agents that are suspected of inhibiting STAT6(B) binding. For example, in one embodiment, molecules from a library of small molecules are used to form a reaction mixture comprising a small molecule and STAT6(B), or an active fragment thereof, and LITAF, or an active fragment thereof. Binding of STAT6(B) with LITAF is then determined via, for example, electrophoresis, Western blotting, etc., and compared to controls wherein no small molecule was added to the reaction mixture and wherein a decrease in binding is indicative of an inhibition in the binding of STAT6(B) with LITAF.

The present invention relates to the interaction of LITAF with the oncogene product p53. Human p53 is a 393-amino acid nuclear transcription factor. Although the present invention is not limited to any particular theory, it is believed that after binding specifically to the promoter regions of its target genes, p53 activates their expression. It is shown in the present invention that p53 directly binds the LITAF promoter region. This binding significantly inhibits LITAF promoter activity and, therefore, suppresses LITAF expression. Therefore, one aspect of the present invention relates to the screening of molecules that may inhibit the binding of p53 to the LITAF promoter. For example, in one embodiment, molecules from a library of small molecules are used to form a reaction mixture comprising a small molecule and p53 (or an active fragment thereof) and a DNA sequence comprising the LITAF promoter region. The mixture is incubated for a length of time and under conditions appropriate for the binding of p53 to the LITAF promoter region. Next, the binding of p53 to the LITAF promoter region is determined (e.g., by electrophoresis or Western blotting). The amount of binding is compared to an identical reaction mixture wherein the small molecule suspected of inhibiting p53 binding to the LITAF promoter region was not added, wherein the decrease in binding in the reaction mixture is indicative or the inhibition of the binding of p53 to the LITAF promoter region.

The present invention also relates to methods of identifying compounds or molecules characterized by the ability to inhibit STAT6(B)/LITAF interaction. For example, one embodiment of such a method comprises screening a library of small molecules for the ability to inhibit STAT6(B)/LITAF interaction by forming a reaction mixture comprising a small molecule to be screened for the ability to inhibit STAT6(B)/LITAF interaction and a mixture of STAT6(B) (or an active fragment thereof) and LITAF (or an active fragment thereof). The mixture is then incubated for a period of time and under conditions appropriate for STAT6(B)/LITAF interaction (e.g., pH 7.0, 37° C. for 5 minutes or pH 7.0, 4° C. for 1 hour). The extent of STAT6(B)/LITAF interaction following the incubation of the mixture by comparing the amount STAT6(B)/LITAF interaction to the amount of STAT6(B)/LITAF interaction detected in an otherwise identical incubation mixture which does not include a small molecule to be screened for the ability to inhibit STAT6(B)/LITAF interaction. In this exemplary assay a decrease in the interaction of the test mixture to that of the otherwise identical incubation mixture is indicative of the ability to of the small molecule to inhibit STAT6(B)/LITAF interaction.

STAT6(B)/LITAF interaction can then be detected by methods known in the art including electrophoresis, electromobility shift assay (EMSA), Western blotting, etc.

The present invention also relates to compounds and methods for the stimulation of angiogenesis by administering to an organism a suitable amount of the compound identified by the above or similar assay or a compound identified or suspected of inhibiting STAT6(B)/LITAF interaction by other methods known in the art. Such identified compounds may be administered either locally or systemically and may be administered as liquids, pills injections, etc. Suitable excipients may also be added to the identified compound(s) for ease of manufacture, stability, ease of administration, etc. Likewise, in one embodiment, the present invention contemplates the stimulation of angiogenesis by the administration to an organism of a compound capable of inhibiting the interaction of STAT6(B) with LITAF to the extent necessary to create a measurable stimulation of angiogenesis.

In yet another embodiment, the present invention also relates to methods of identifying compounds or molecules characterized by the ability to stimulate STAT6(B)/LITAF interaction. For example, one embodiment of such a method comprises screening a library of small molecules for the ability to stimulate STAT6(B)/LITAF interaction by forming a reaction mixture comprising a small molecule to be screened for the ability to stimulate STAT6(B)/LITAF interaction and a mixture of STAT6(B) (or an active fragment thereof) and LITAF (or an active fragment thereof). The mixture is then incubated for a period of time and under conditions appropriate for STAT6(B)/LITAF interaction (e.g., pH 7.0, 37° C. for 5 minutes or pH 7.0, 4° C. for 1 hour). The extent of STAT6(B)/LITAF interaction following the incubation of the mixture by comparing the amount STAT6(B)/LITAF interaction to the amount of STAT6(B)/LITAF interaction detected in an otherwise identical incubation mixture which does not include a small molecule to be screened for the ability to stimulate STAT6(B)/LITAF interaction. In this exemplary assay an increase in the interaction of the test mixture to that of the otherwise identical incubation mixture is indicative of the ability to of the small molecule to stimulate STAT6(B)/LITAF interaction.

STAT6(B)/LITAF interaction can then be detected by methods known in the art including electrophoresis, electromobility shift assay (EMSA), Western blotting, etc.

The present invention also relates to compounds and methods for the inhibition of angiogenesis by administering to an organism a suitable amount of the compound identified by the above or similar assay or a compound identified or suspected of stimulating STAT6(B)/LITAF interaction by other methods known in the art. Such identified compounds may be administered either locally or systemically and may be administered as liquids, pills injections, etc. Suitable excipients may also be added to the identified compound(s) for ease of manufacture, stability, ease of administration, etc. Likewise, in one embodiment, the present invention contemplates the stimulation of angiogenesis by the administration to an organism of a compound capable of stimulating the interaction of STAT6(B) with LITAF to the extent necessary to create a measurable stimulation of angiogenesis.

Furthermore, the present invention relates to a method for identifying a small molecule characterized by the ability to inhibit STAT6(B)/VEGF promoter interaction. For example, one embodiment of such a method comprises screening a library of small molecules for the ability to inhibit STAT6(B)/VEGF promoter interaction by forming a reaction mixture comprising a small molecule to be screened for the ability to inhibit STAT6(B)/VEGF promoter interaction and a mixture of STAT6(B) (or an active fragment thereof) and the VEGF promoter. The mixture is then incubated for a period of time and under conditions appropriate for STAT6(B)/VEGF promoter interaction (e.g., pH 7.0; 37° C. for 5 minutes or pH 7.0, 4° C. for 1 hour). The extent of STAT6(B) VEGF promoter interaction following the incubation of the mixture by comparing the amount STAT6(B)/VEGF promoter interaction to the amount of STAT6(B)/VEGF promoter interaction detected in an otherwise identical incubation mixture which does not include a small molecule to be screened for the ability to inhibit STAT6(B)/VEGF promoter interaction. In this exemplary assay a decrease in the interaction of the test mixture to that of the otherwise identical incubation mixture is indicative of the ability to of the small molecule to inhibit STAT6(B)/VEGF promoter interaction.

The STAT6(B)/VEGF promoter interaction of the example given above may be detected by a method selected from a group consisting of for example, electrophoresis, electromobility shift assay (EMSA) and Western blot.

While specific embodiments of the present invention have been described, it will be apparent to those skilled in the art that various modifications thereto can be made without departing from the spirit and scope of the invention as defined in the examples and appended claims.

### EXAMPLES

### Example 1

### Identification of a Novel Protein, STAT6(B) that Interacts with LITAF

A yeast two-hybrid system was used to identify mediators that might interact with LITAF in the regulation of TNFα. First, LITAF full length DNA was inserted into expression plasmid pGBKT7, with the resulting construct named pGBKLITAF. This recombinant DNA containing a selection marker (-Trp) was used as the bait, and a human spleen pACT2-cDNA library with a different selection marker (-Leu) was used for the activation domain (AD) fusion constructs. Both bait and AD DNAs were mixed and transformed into yeast AH109 cells. In order to eliminate false positives, high-stringency conditions were used to select transformants on SD/-Trp/-Leu plus -Ade/-His medium. The transformants surviving in high-stringency medium were screened and only the AD DNA from the transformants was prepared. The DNA was re-confirmed by retransformation and re-hybridization with pGBKLITAF in AH109 cells. A clone was isolated after high-stringency screenings. Sequence analysis of this clone showed that it contained an open reading frame (ORF; 1404 aa) and a poly A tail (FIG. 1a). The DNA sequence and its ORF in the region from 151 to 404 aa were homologous to a known gene, STAT6 (NM_{—}903153, GenBank), referred to as STAT6(A) in this study, except for two amino acid differences: P-S at aa position 292 and M-T at aa position 324. However, the sequence in the region from 1 to 150 aa was completely different from STAT6(A), as shown in FIG. 1b. The 3′ untranslated region (UTR) of this clone, from the stop codon (1,408 bp) to poly A tail, (2,141 bp) was almost identical with the UTR of STAT6(A), with only three differences: base pairs were shifted: c-g at 1906 bp and g-a at 1935 bp, and a single deletion of an a base pair (2085 bp to 2086 bp, FIG. 1a).

To confirm the difference between STAT6(A) and STAT6(B), Northern blots were prepared with RNA samples from multiple human tissues and probed with both transcripts. As seen in FIG. 1c, distinct transcripts of approximately 3.5 kb for STAT6(A), and of approximately 1.5 kb for STAT6(B) were identified. Although both were abundantly expressed in spleen tissue, STAT6(B) transcripts were more highly expressed in samples prepared from colon, testis and peripheral blood leukocytes in comparison with STAT6(A). STAT6(B) chromosomal localization was then determined by in situ hybridization. The fluorescent banding spots were observed on chromosome 12q13 that is close to the location of STAT6(A) (Xie, et al., Eur J Immunol, 32:2837-2846, 2002).

Investigation of the Interaction Between LITAF and STAT6(B) by Immunoprecipitation.

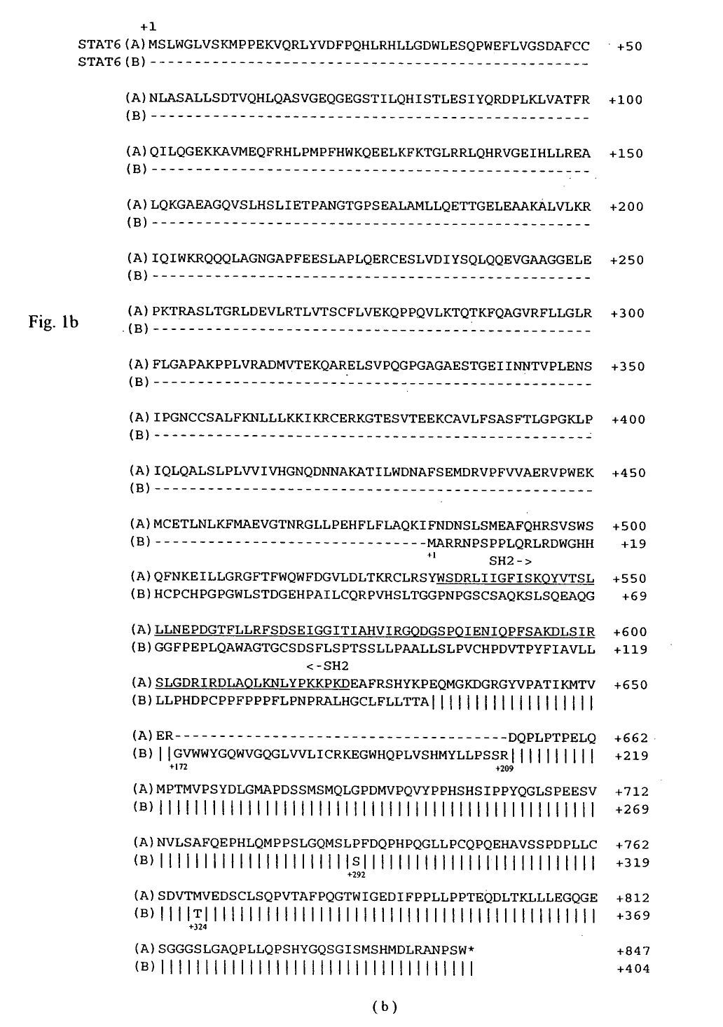

Although yeast two hybrid experiments showed an association between LITAF and STAT6(B), more definitive evidence about their interaction is required. Thus, mouse macrophages were used for immunoprecipitation experiments designed to determine whether LITAF interacts with STAT6(B). STAT6 antibody was used to detect the product of STAT6(A) (120 kD) or STAT6(B) (approximately 50 kD) because the antibody specifically recognizes the carboxyl terminus of STAT6(A) of mouse or human, which is also shared by STAT6(B). Additionally, the antibody (Stat6, Santa Cruz) originally made to bind to STAT6(A) would be expected to bind murine STAT6(B) because the carboxyl terminus of STAT6(B) has more than 88% homology to mouse STAT6 (A) (L47650, GenBank). The cell lysates extracted from LPS-treated mouse macrophages were immunoprecipitated with the appropriate antibody followed by Western blotting. As shown in FIG. 2, lysates pulled down with either LITAF or STAT6(B) could be completely detected by LITAF or STAT6(B) antibody (FIG. 2, lanes 4 and 6). In FIG. 2, lanes 1, 3 and 5 were untreated and lanes 2, 4 and 6 were treated with 100 ng/ml of LPS. Lanes 3 and 4 were immunoprecipitated with anti-LITAF and lanes 5 and 6 were immunoprecipitated with anti-STAT6. These findings suggest that full-length LITAF directly interacts with full-length STAT6(B). Interestingly, the result (FIG. 2) shows that both LITAF and STAT6(B) are induced specifically by LPS. In addition, mouse STAT6(A) could not be detected in either non-LPS-treated or LPS-treated lysates (FIG. 2, lanes 1 and 2); therefore, the effects on mouse STAT6(A) appear not to be relevant in this pull-down assay, and was not considered further.

LPS-Dependent STAT6(B) Gene Expression.

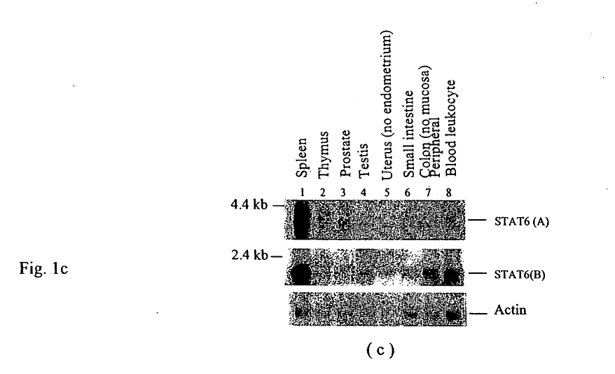

Since STAT6(B) was identified as involved in interaction with LITAF and both proteins were clearly observed after LPS stimulation in mouse macrophages (FIG. 2), it was hypothesized that human STAT6(B) gene expression is also involved in LPS-dependent regulation of cytokines as well as LITAF. In order to test this hypothesis, matured THP-1 cells were stimulated with various doses of LPS, then cell lysate was harvested and subjected to Western blot analysis with the indicated antibodies: Stat6 (S-20), LITAF (611615) or Actin (C-11) as control. As shown in FIG. 3, the protein level of STAT6(B) or LITAF was progressively increased in response to increasing doses of LPS (lane 1 was untreated, lanes 2-6 were pretreated with 5, 25, 50, 100 or 200 ng/ml LPS for 3 hours, respectively). In contrast, STAT6(A) production was not significantly changed in response to LPS concentration. This result strongly suggests that both LITAF and STAT6(B), but not STAT6(A), are induced in an LPS-dependent manner.

EMSA (Electrophoretic Mobility Shift Assay).

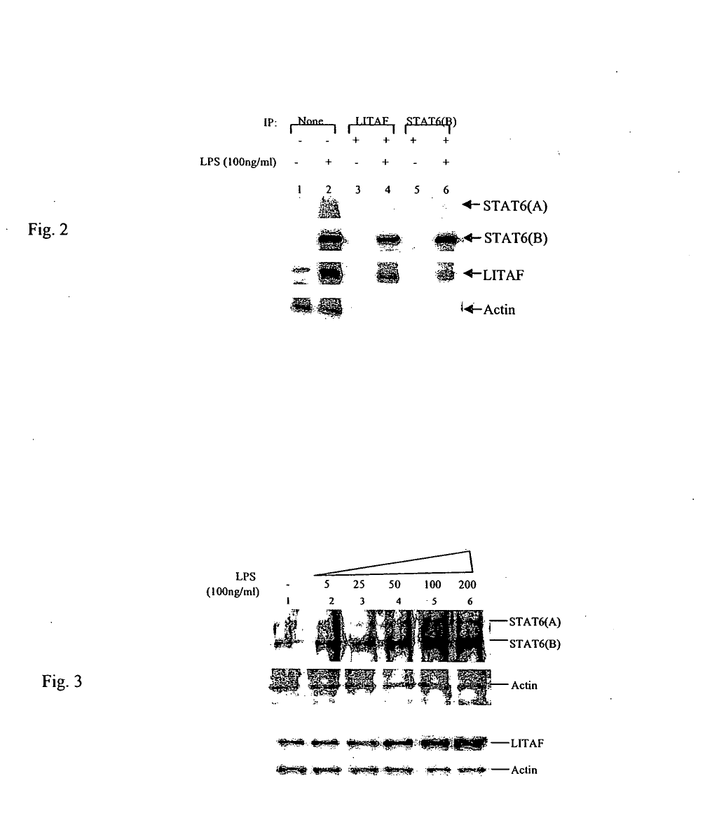

To determine whether STAT6(B) contains a binding domain for LITAF, EMSA was performed. For the EMSA shown in FIG. 4, protein (30 μg) was extracted from each culture of transfected U²OS cells and was added to appropriate reaction buffer with the oligo DNA probe, [³²P] ATP-labeled hTNF-α promoter DNA. Protein from untranscribed cells was applied in lanes 1 and 2 plus a 50-fold excess of unlabeled competitor (lane 2). The proteins from each condition of cells treated with 1 μg DNA of empty vector alone (lane 3), full length of LITAF alone (lane 4), both full length LITAF and STAT6(A) (lane 5), full length LITAF plus a fragment of STAT6(B) (aa 1-150, lane 6), full length LITAF plus a fragment of STAT6(B) (aa 151-404, lane 7 and full length LITAF plus full length STAT6(B) (aa 1-404, lane 8) were assessed by EMSA. The shifted DNA bands are indicated by arrows. DNAs of full length LITAF (FIG. 4, lanes 4-8), empty vector 11 (lane 3), STAT6(A) (lane 5), and STAT6(B) fragment (lanes 6-8) were transfected or co-transfected into U₂OS cells. The resulting cell extracts were then purified and assessed by EMSA as described below. The shifted protein bands shown in FIG. 4 indicated that STAT6(B) contains a LITAF-binding domain in the region from 1 to 150 aa. This result suggests that overexpression of both LITAF and STAT6(B) leads to formation of a LITAF-STAT6(B) heterodimer complex that can bind to the TNF promoter.

Translocation of LITAF/STAT6(B) Complex.

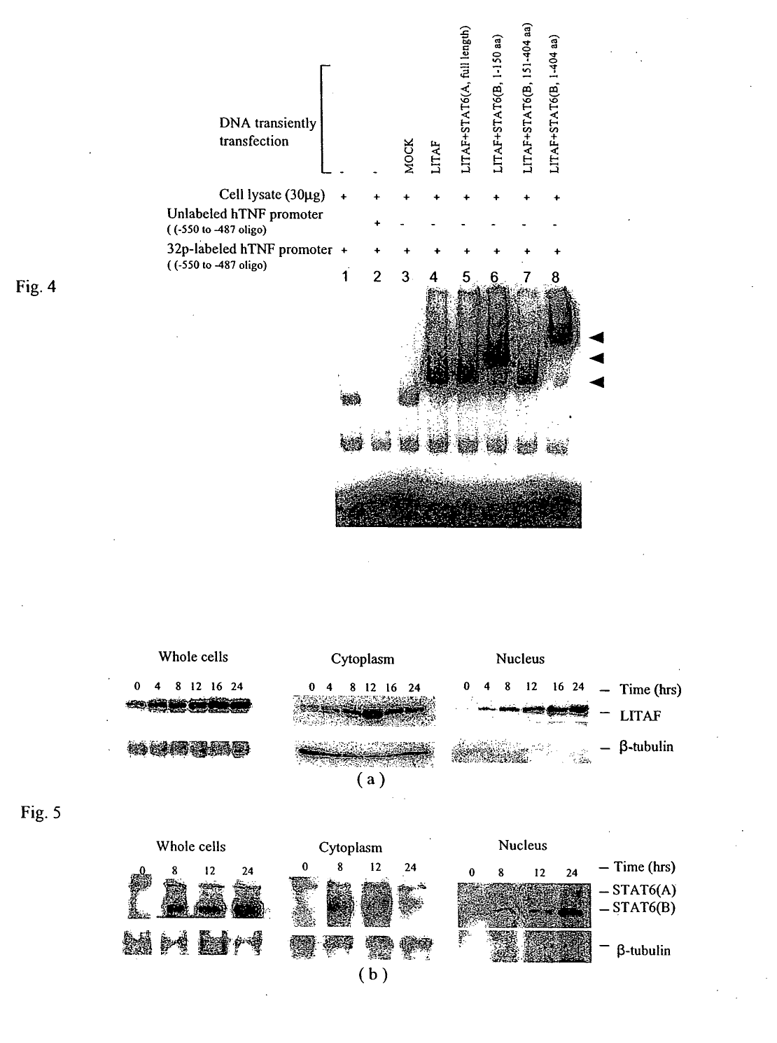

Homodimers of STAT6(A) are known to translocate from cytoplasm to nucleus, where they bind to target gene sequences and activate the expression of those genes-(Scott, et al, Clin Diagn Lab Immunol, 9:1153-1159, 2002). Since the newly identified STAT6(B) was found to have a high degree of homology at the carboxy terminus with STAT6(A), it was hypothesized that a LITAF-STAT6(B) complex might also translocate into the nucleus after formation in cytoplasm. Thus, fluctuations in protein levels of STAT6(B) and LITAF were measured over time in cells known to produce TNF in a regulated manner. DNAs of both STAT6(B) (full length) and LITAF (full length) were co-transfected into THP-1 monocytic cells. Cell extracts harvested at various times after transfection were purified and subjected to Western blot analysis. Protein extracts were collected from whole cells, cytoplasm or nuclei after overexpression of both LITAF and STAT6(B). As seen in FIG. 5, the protein levels of both LITAF (FIG. 5a) and STAT6(B) (FIG. 5b) gradually rose in both whole cell extracts and nucleus at a similar rate. In contrast, 24 hours post-transfection the amount of cytoplasmic STAT6(B) decreased sharply and the level of cytoplasmic LITAF dropped approximately 2 fold. It was observed that both LITAF and STAT6(B) levels increased steadily in the nucleus after that time. This increase of LITAF and STAT6(B) levels in the nucleus coincided with a decline in their concentrations in the cytoplasm. These changes over time suggest that a LITAF-STAT6(B) protein complex may be forming and translocating into the cell nucleus 4 to 8 hrs after transfection. Interestingly, STAT6(A) did not seem to be affected by overexpression of LITAF because its concentration was constant in both whole cell extracts and cytoplasm. In addition, STAT6(A) protein could not be found in the nucleus at any of the times selected. Finally, the absence of β-tubulin control from nuclear samples indicated that the nuclear extracts were not contaminated with proteins from the cytoplasm during the protein purification process (Leonard, Nat Med, 2:968-969, 1996).

Regulation of Cytokines by LITAF/STAT6(B) Complex.

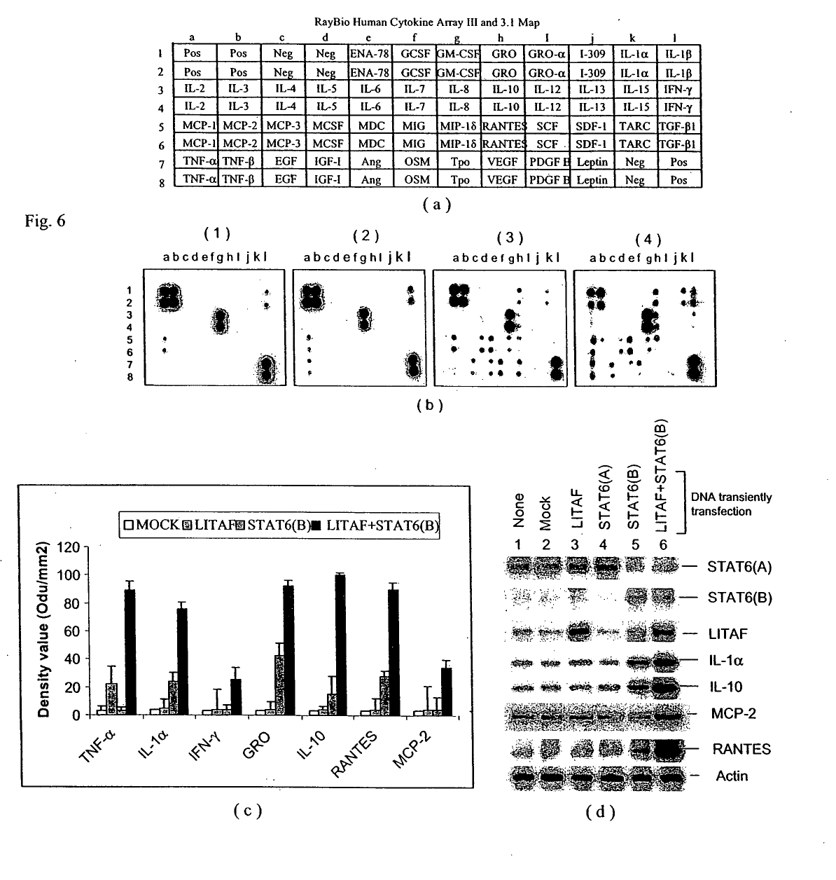

Since both LITAF and STAT6(B) were found in the nucleus (FIG. 5), it was further investigated whether they act separately or in concert in mediating activation of TNF-α. While studying the complex, a cytokine array was employed to look at effects on other cytokines (FIG. 6a-6c.). The assay was preformed as follows. Using an empty vector as a control (FIG. 6b1), DNA of LITAF (full length, FIG. 6b2), STAT6(B) (full length; FIG. 6b3) or both full length of LITAF+STAT6(B) (FIG. 6b4), respectively, was transfected or co-transfected into THP-1 cells. Conditioned medium from each treated cell type was then prepared and incubated with membranes containing an array of 44 human protein cytokine antibodies (FIG. 6a). Autoradiographs were scanned, and the density of each cytokine at the corresponding position was determined. Each blot shown in FIG. 6b shows a single result that is representative of three replicates. The relative intensities of each cytokine were normalized to control spots on the same membrane. Major increases or decreases among cytokines are represented graphically in FIG. 6c. LITAF alone (FIG. 6b2) modestly up-regulated TNF-α (positions a-7, a-8) and IL-1α (positions 1-1, 1-2). STAT6(B) alone (FIG. 6b3) up-regulated several different target cytokines such as IL-1α (k-1, k-2), GRO (h-1, h-2), IL-10 (h-3, h-4) and RANTES (h-5, h-6). Interestingly, the cytokines that were induced only modestly by either LITAF or STAT6(B) alone were more significantly up-regulated in the cells co-transfected with both LITAF and STAT6(B) (FIG. 6b4): 2.1 fold for GRO, 3 fold for IL-1α 3.2 fold for RANTES, 4 fold for TNF-α, 6.2 fold for IFN-γ, 8.5 fold for MCP-2 and 10 fold for W-10. In order to confirm these findings at the protein level we selected the four most strongly affected cytokines, IL-1α, IL-10, MCP-2 and RANTES and, under the same experimental conditions, measured their protein levels by Western blots. As shown in FIG. 6d, these protein levels were not altered in response to overexpression of LITAF alone (lane 3) or of STAT6(A) alone (lane 4). However, modest induction of all except MCP-2 was observed after transient transfection of STAT6(B) alone (lane 5) while the combination of LITAF and STAT6(B) resulted in big increases in all of the 13 cytokines tested (lane 6). These results are consistent with the array data shown in FIG. 6, although we note that the other RNA fluctuations observed in the array have not yet been confirmed by Western blot analysis.

Silencing of LITAF Expression by LITAF-Specific shRNA.

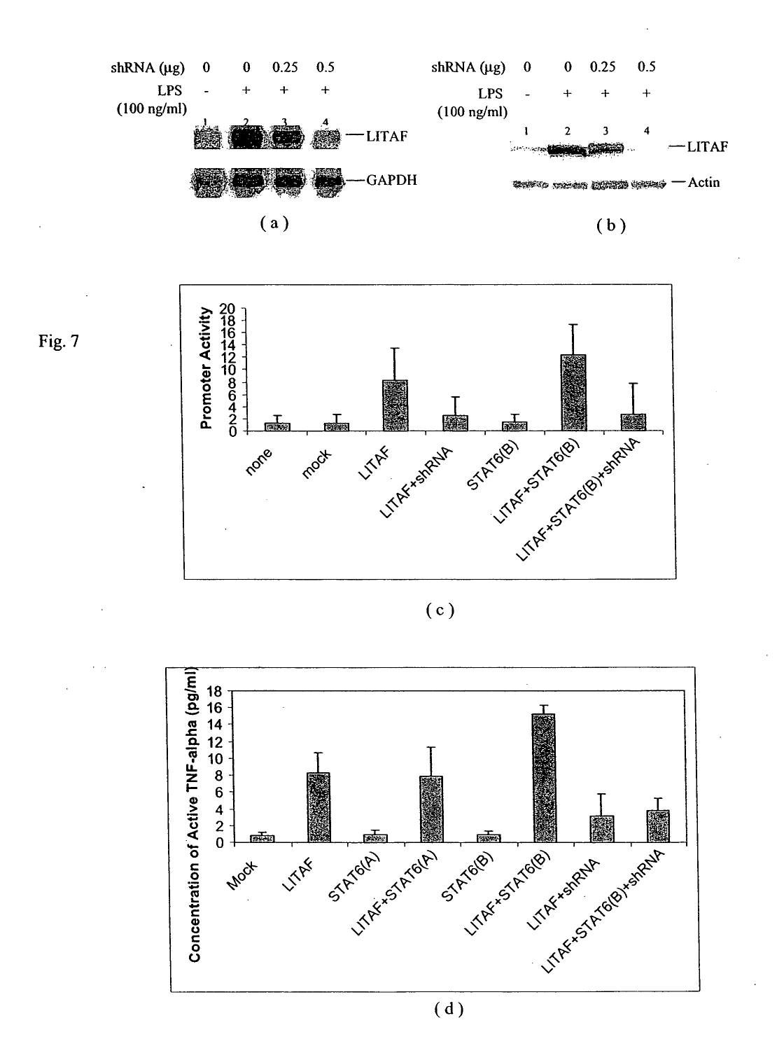

Since the LITAF/STAT6(B) putative complex showed a greater capacity to regulate target cytokines in comparison with either LITAF or STAT6(B) alone, it was considered that LITAF plays an important regulatory role in the complex. As a first step, it was investigated if LITAF could be silenced by delivery of LITAF-specific shRNA. Northern blot analysis was performed on total RNA extracted from LPS-stimulated THP-1 cells after transfection with 0.25 μg or 0.5 μg of pSHAG-LITAF (pSHAG: Short Hairpin Activated Gene silencing). Meanwhile, proteins from each of the treated cell cultures were purified and subjected to Western blot analysis. As shown in FIGS. 7a and 7b, both the RNA level (FIG. 7a, lane 4), and the protein level (FIG. 7b, lane 4) of LITAF induced by LPS were greatly decreased in cells transfected with 0.5 μg/ml of pSHAG-LITAF, demonstrating that the shRNA driven by pSHAG-LITAF effectively silenced LITAF gene expression.

In order to characterize the functional importance of LITAF in the LITAF/STAT6(B) complex, the effect of the complex was examined, with and without silenced LITAF, on TNF-α promoter activity. U₂OS cells containing the TNF-promoter linked to luciferase were transiently transfected with LITAF alone, STAT6(B) alone, or both DNAs as described. TNF-α promoter activity was analyzed via luciferase activity. As shown in FIG. 7c, the LITAF-STAT6(B) co-transfection caused a 1.5 fold increase in TNF-promoter activity, in comparison to transfection with LITAF alone. STAT6(B) alone did not substantially change luciferase activity. However, the ability of either LITAF alone or LITAF-STAT6(B) complex to increase TNF-promoter activity was significantly inhibited by LITAF-shRNA. The TNF-α concentrations in the supernatants of these transfected THP-1 cells was also measured. As shown in FIG. 7d, 1.8-fold more TNF-α was produced after 16 hours by cells containing the LITAF-STAT6(B) complex than by cells transfected with LITAF alone. The level of secreted TNF-α protein induced by either LITAF alone or LITAF-STAT6(B) complex was also significantly decreased by the delivery of shRNA in cells. These findings confirm, both by luciferase assay and ELISA, that shRNA introduced to silence the effect of LITAF in the LITAF-STAT6(B) complex resulted in a decrease of TNF-α gene expression and protein production. They further indicate that LITAF might play a leading role in cytokine regulation after the LITAF-STAT6(B) complex translocates to the nucleus in the cells.

### Example 2

### Generation of LITAF-Deficient and STAT6(B)-Deficient Animals

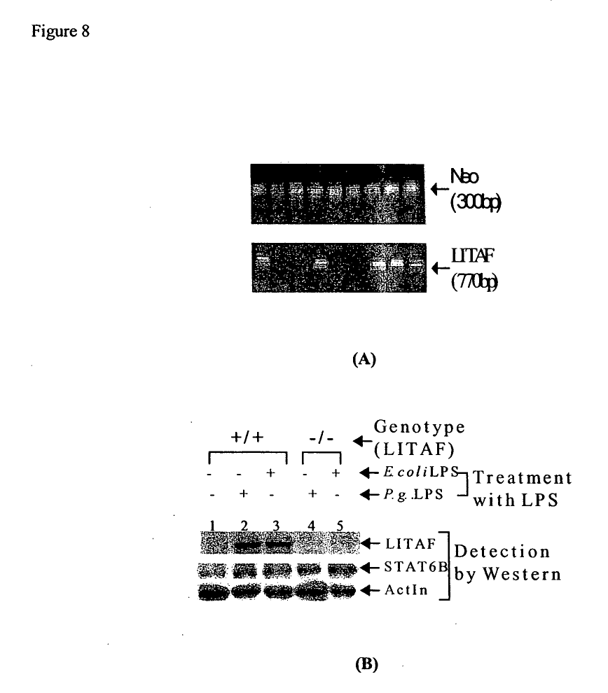

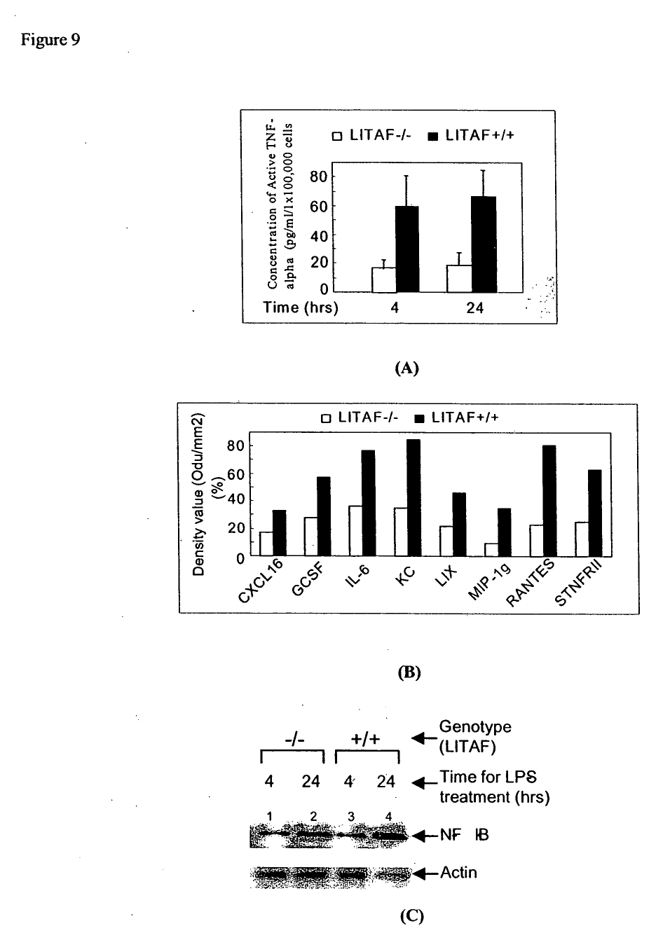

LITAF heterozygotes (LITAF−/+×LITAF−/+) were crossed to obtain LITAF−/− homozygote mice. Genotyping was performed by PCR (FIG. 8a) and confirmed by Western blot analysis (FIG. 8b). Mice containing neomycin but lacking the LITAF gene were considered homozygotes (FIG. 8). Furthermore, homozygous mice showed no LITAF protein expression by Western blot (FIG. 8b). To define the phenotype of LITAF−/− mice, levels of TNF protein were assayed on peritoneal macrophages extracted from LITAF−/− and wild-type mice and exposed to LPS. As expected, levels of TNF were 3-fold lower in LITAF−/− macrophages than in wild-type macrophages after LPS stimulation (FIG. 9a). The TNF-concentrations shown are the averages of 3 independent experiments from 3 sets of animals.

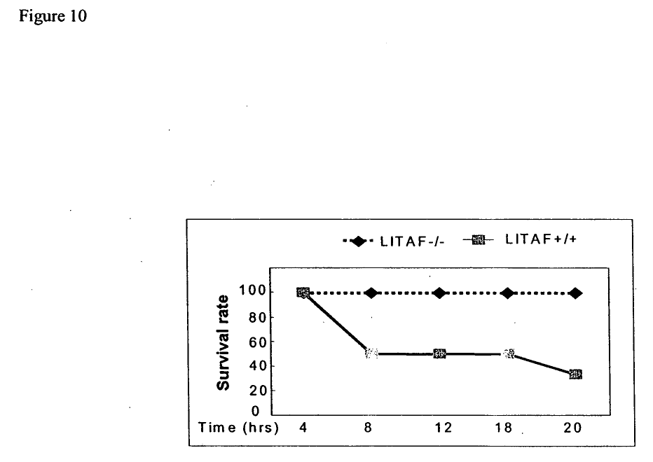

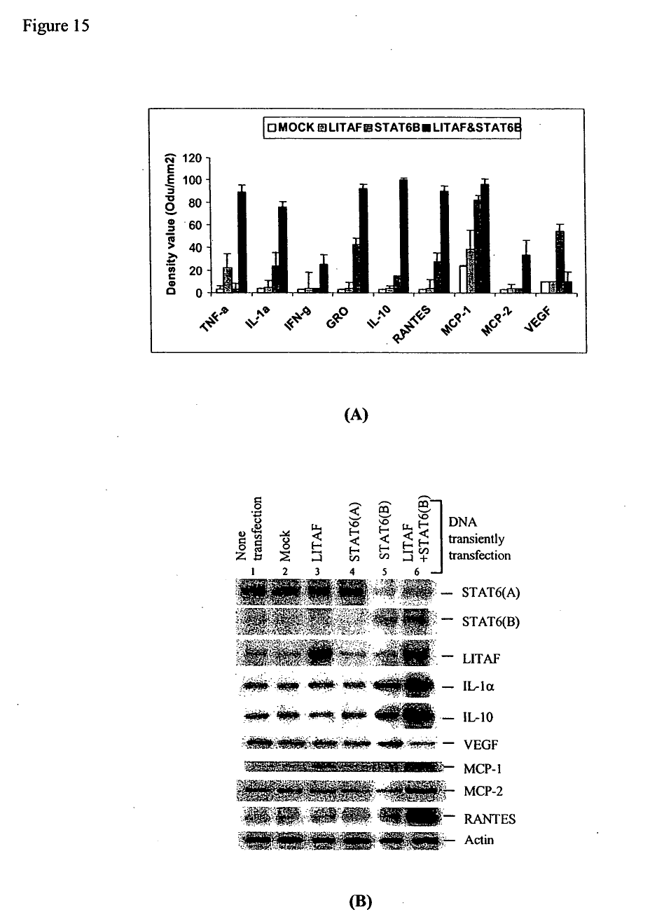

To extend our results to other cytokines, a cytokine antibody array similar to the one employed in FIG. 15a, below, was used with the same macrophages extracts and increases or decreases among cytokines are represented graphically in FIG. 9b. The density value of each test sample was normalized to the positive control from the same lysates and plotted. All of the cytokines tested exhibited a substantial reduction of expression in LITAF−/− macrophages compared to the wild-type macrophage. Of note was that soluble TNFRII (sTNFRII) expression was found significantly suppressed in LITAF−/− probably due to the reduced TNF-secretion (FIG. 9a). This is probably because sTNFRII expression is known to be dependent on TNF-α. To test whether NFkB was disregulated as a result of LITAF deficiency, levels of NFkB were detected in these macrophages and found similar in homozygotes and wild-type (FIG. 9c). These results demonstrate that LITAF deficiency did not perturb NFkB expression and the reduced cytokine expression observed after LPS stimulation in these mice can probably be attributed to LITAF deficiency. Finally, we performed in vivo LPS lethality experiments in LITAF−/− animal. In murine models it is well accepted that D-galactosamine (D-GalN) dramatically sensitizes mice to the lethal effects of LPS as the result of its toxic effects on hepatocytes. There is agreement that death in LPS/D-GalN-challenged animals is due to TNF toxicity. After intraperitoneal injection of each mouse with 250 ng of E. coli UPS plus 25 mg D-GalN, the animal's condition was closely monitored. As shown in FIG. 10, LITAF−/− were more resistant to LPS-induced lethality than LITAF+/+ mice with a survival rate up to 20 hours after LPS injection to be almost 100% while only 30% of LITAF+/+ animals survived. Altogether these data strongly substantiate our in vitro data and provide a phenotype for the LITAF−/− animals instrumental for our studies. STAT6(B)-deficient and LITAF-STAT6(B)-deficient animals are made and confirmed similarly.

### Example 3

### Investigation of p53/LITAF/TNF Pathway

### The Role of p53

It was observed that LITAF may be linked to the well known sensor and mediator of damage-induced apoptosis, p53 (Polyak, et al., Nature, 389:300-305, 1997, which is herein incorporated by reference). Human p53 is a 393-amino acid nuclear transcription factor. After binding specifically to the promoter regions of its target genes, p53 activates their expression. This example determined the nature of the signaling involved in p53 accumulation and LITAF mRNA induction after LPS stimulation. It was demonstrated that a direct binding between p53 and LITAF promoter took place. The region on the LITAF promoter where p53 binds was also identified. The specific site required for p53 binding is located between −600 to −480 bp on the LITAF promoter and binding of p53 protein significantly inhibits LITAF promoter activity. These findings show that LITAF might be indirectly induced by p53, which implies the involvement of other factor(s) in direct induction of LITAF or that p53 represses LITAF.

### Example 4

### LITAF-STAT6(B) Complex Involvement in VEGF Mediated Angiogenesis and Tubulogenesis

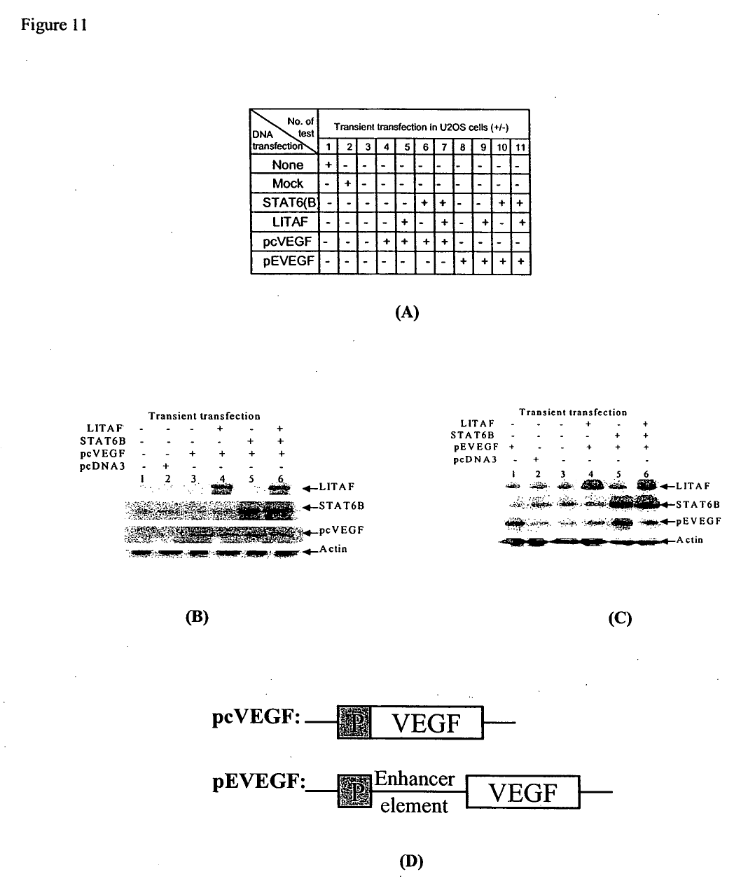

These data showed that overexpression of STAT6(B) affects VEGF protein expression. To determine that this activation occurred at the transcriptional level, VEGF promoter activity assays were performed. A full length VEGF sequence, with or without its enhancer element (−2040˜+1), named pcVEGF or pEVEGF, was cloned into pcDNA3 plasmid. The cloned DNA with LITAF and/or STAT6(B) DNAs were transiently co-transfected into U₂OS cells as described in FIG. 11a. As shown in FIG. 11b+c, the transient transfection of STAT6(B) alone into these cells significantly upregulated VEGF gene expression via its enhancer element (FIG. 11d). However, transfection of LITAF alone did not have any effect while the co-transfection of STAT6(B) and LITAF suppressed VEGF gene transcription.

In Vitro Angiogenesis/Tubulogenesis assay of VEGF. To expand the observations that STAT6(B) is a potent stimulator of VEGF, and that the LITAF-STAT6(B) complex is inhibitory, culture supernatant fluids of LITAF-, STAT6(B)- or LITAF-STAT6(B)-transfected THP1 cells were tested in an in vitro angiogenesis/tubulogenesis assay. The assay was preformed as follows. Porcine aortic endothelial (PAE) cells expressing VEGFR-2 spheroids were generated as described in the art (Mayer and Rahimi, Ann. NY Acad. Sci, 995:200-207, 2003). A defined number of cells were suspended in DMEM containing 1% fetal bovine serum and 0-0.24% (w/v) carboxymethylcellulose (4000 centipoise) in non-adherent round-bottom 96-well plates under standard cell culture conditions. After 24 hours all cells formed one single spheroid per well (about 750 cells/spheroid). Spheroids were cultured for 2 days before using them in the in vitro angiogenesis assay in the following manner Spheroids containing about 750 cells were embedded in collagen gels. Eight volumes of collagen were mixed with 1 volume of 10×HEPES-buffered saline solution containing 10% 10×DMEM with phenol red. Spheroids were centrifuged and suspended in 9 ml of DMEM containing 0.96% carboxymethylcellulose. Collagen and spheroids were mixed and transferred to prewarmed 24-well plates and the gels were allowed to polymerize in the incubator. After 30 min, 100 μl of DMEM containing various conditioned medium were added to top of the gel. After 2 days, sprouting and tubulogenesis were observed with an inverted phase-contrast microscope and pictures were taken using the SPOT camera system.

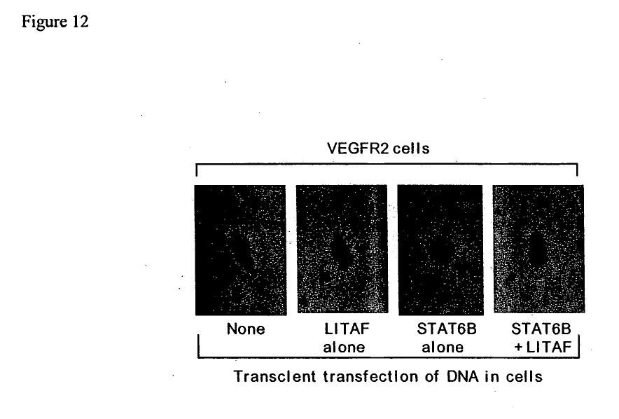

It was determined whether VEGF, induced by STAT6(B), was active and capable of inducing angiogenesis and that the LITAF-STAT6(B) complex effect would inhibit this activity. LPS-induced THP1 served as control. As shown in FIG. 12, supernatant from STAT6(B) transfected THP-1 cells stimulated in vitro angiogenesis/tubulogenesis while the effects of the complex on VEGFR2 cells was similar to the control. This data confirms observations that VEGF is stimulated by STAT6(B) while the STAT6(B)/LITAF complex inhibits it.

### Example 5

### Characterization of LITAF and STAT6(B) Phosphorylation and Dephosphorylation Processes and Determine their Involvement in Cytokine Gene Regulation

The data of this prophetic Example will show that phosphorylation of STAT6(B) and LITAF might be affected by p38 MAP kinase while protein tyrosine phosphatase (PTP), which binds LITAF, may act in the dephosphorylation of the LITAF-STAT6(B) complex. Thus, (a) the kinase(s) (p38MAPK as well as other potential kinases) may be involved in the phosphorylation of STAT6(B) and LITAF; (b) phosphorylated STAT6(B) or LITAF forms homodimers or heterodimers to regulate cytokines; (c) the dephosphorylation process of LITAF-STAT6(B) and the role of PTP are investigated and; (d) inflammatory cytokines involved in LITAF and STAT6(B) processes can be modulated by these kinases and phosphatases.

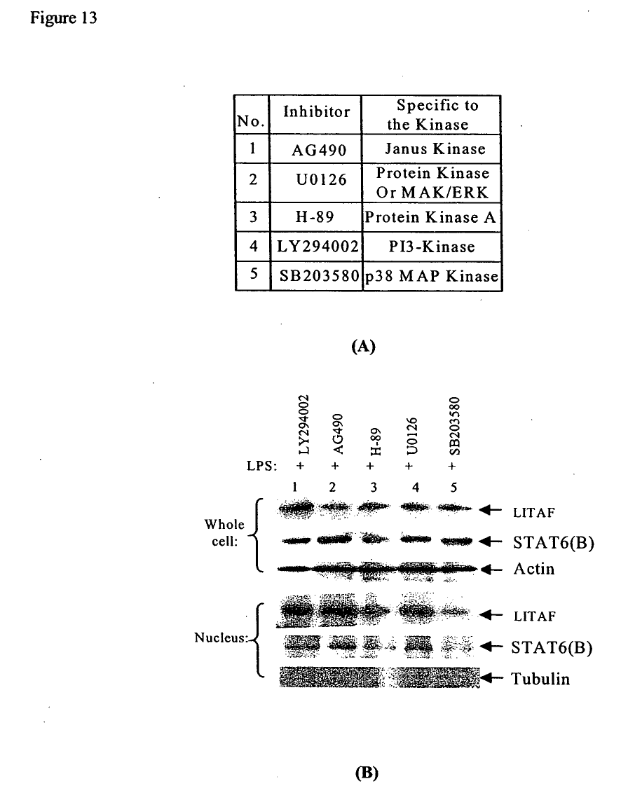

It is well known that STAT family members share two major domains, oligomerization and Src homology 2 (SH2), for phosphorylation by Janis kinases (Jaks) that facilitate dimerization of STAT proteins and subsequent translocation into the nucleus (Gallmeier, et al., J Cell Physiol, 203:209-216, 2005; Chen, et al., J Allergy Clin Immunol, 114:476-489, 2004, Tang, et al., PNAS USA, 102:5132-5137, 2005). However, STAT6(B) does not seem to be phosphorylated by Jaks as it lacks both an oligomerization and SH2 domain, suggesting that it must be phosphorylated by another kinase. The data in FIG. 13 indicates that p38 MAP kinase (lane 5) phosphorylates STAT6(B) and LITAF. In this Western blot, cells were pretreated with various kinase inhibitors (FIG. 13a) before LPS stimulation. The p38MAPK inhibitor SB203580 substantially reduced both LITAF and STAT6(B) expression in the nucleus while whole cell extract LITAF and STAT6(B) expression was found unchanged. This inhibition resulted in a suppression of TNF-α protein levels compared to cells exposed to LPS only (FIG. 13b).

This experiment will show that LPS-induced STAT6(B) and/or LITAF protein is/are phosphorylated by p38MAPK producing a complex that is translocated into the nucleus to activate target cytokine gene expression such as TNF-α or MCP-1. This translocation and further activation of gene expression is prevented when PTP dephosphorylates the complex in cytoplasm. This alteration of the protein levels in nucleus is measured by Western blot and the consequence on TNF-α and MCP-1 secretion by ELISA. Finally, kinase and phosphatase assays further confirm our data.

Plasmid construction. The following full length and specific mutant constructs have been designed and are used for the investigation of the specific binding domain for p38 MAPK phosphorylation and PTP dephosphorylation. (1) Construction of the full length (1,104 bp) human p38MAP kinase DNA (U66243). This is generated by PCR with the primer pairs: 5′-TGCCATGAGCTCTCCGCCGCC-3′ [SEQ ID NO: 5] and 5′-TCACAGAGGCGTCTCCTTGGA-3′ [SEQ ID NO: 6] and subcloned into pcDNA3HA (2) Construction of the full length (447 bp) human PTP (NM_{—}007079). This is generated by PCR with the primer pairs: 5′-ATGGCTCGGATGAACCGCCCG-3′ [SEQ ID NO: 7] and 5′-CTACATAACGCAGCACCGGGT-3′ [SEQ ID NO: 8] and subcloned into pcDNA3HA. (3) pcDNAHA-STAT6(B) expresses full length STAT6(B) protein in mammalian cells, wtTNFP (−991 to 1) or mtTNFP 1 (−991 to 1Δ-515 to -511) contains full length or deletion of TNF-α promoter for the luciferase assay, respectively (Tang, et al., PNAS USA, 100:4096-4101, 2003; Tang, et al., PNAS USA, 102:5132-5137, 2005). (4) The series of STAT6(B) DNAs will be generated by PCR with primer pairs: (aa1-100) 5′-ATGGCCCGACGGAACCCTTCTC-3′ [SEQ ID NO: 9] and 5′-GAGGGCAGCGGGGAGCAGGGA-3 [SEQ ID NO: 10]; (aa 1-200) 5′-ATGGCCCGACGGAACCCTTCTC-3′ [SEQ ID NO: 11] and 5′-ACTGACCAAGGGTTGATGCCA-3 [SEQ ID NO: 12]; (aa 1-300) 5′-ATGGCCCGACGGAACCCTTCTC-3′ [SEQ ID NO: 13] and 5′-GCCCTGGGGGTGAGGCTGGTC-3 [SEQ ID NO: 14]; (aa 1404) 5′-ATGGCCCGACGGAACCCTTCTC-3′ [SEQ ID NO: 15] and 5′-TCACCAACTGGGGTTGGCCCT-3 [SEQ ID NO: 16], are respectively subcloned into pGEX4T-1 or pcDNAHA plasmid. These clones are used for the in vitro kinase and phosphatase assays. (5) The series of LITAF DNA is generated by PCR with the follow primer pairs: (aa 1-75) 5′-CGGGATCCAT GTCGGTTCCAGGACCT-3′ [SEQ ID NO: 17] and 5′-cggaattcggtattggatttt-3′ [SEQ ID NO: 18]; (aa 1-151) 5′-CGGGATCCATGTCGGTTCCAGGACCT-3-[SEQ ID NO: 19] and 5′-cggaattccagttgggacagtaatgg-3′ [SEQ ID NO: 20]; (aa 76-151) 5′-CGGGATCCGTGCAGACGGTCTACGTG-3′ [SEQ ID NO: 21] and 5′cggaattccagttgggacagtaatgg-3′ [SEQ ID NO: 22]; (aa 1-228) 5′-CGGGATCCATGTCGGTTCCAGGACCT-3′ [SEQ ID NO: 23] and 5′-cgggatcetcagggtctcagggaggc-3′ [SEQ ID NO: 24]; (aa 76-228) 5′-CGGGATCCGTGCAGACGGTCTACGTG-3′ [SEQ ID NO: 25] and 5′-cgggatcctcagggtctcagggaggc-3′ [SEQ ID NO: 26]; (aa 152-228) 5′-CGGGATCCCAGAGCTCTCCTGGGCAC-3′ [SEQ ID NO: 27] and 5′-cgggatcctcagggtctcagggaggc-3′ [SEQ ID NO: 28]; which are respectively subcloned into pGEX4T-1 or pCDNAHA plasmids.

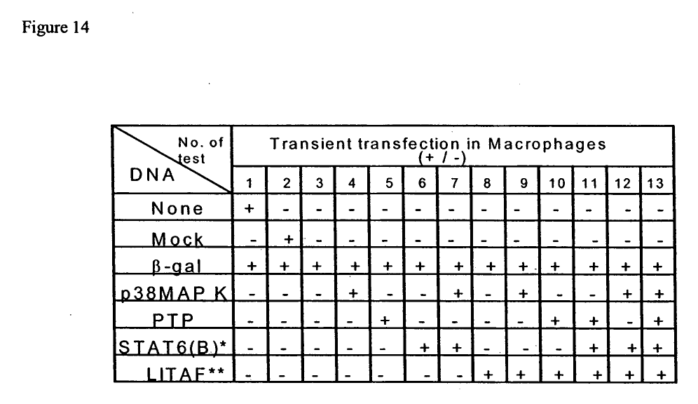

Transient transfection of DNAs in mouse macrophage cells. To investigate whether p38MAP kinase and PTP are regulating the phosphorylation between STAT6(B) and LITAF, p38MAP kinase or PTP are over expressed and the kinase and phosphatase activities evaluated by immunoprecipitation and EMSA. For that, 1×10⁶ wild-type mouse macrophage cells are co-transfected with 1 μg DNAs by Fugene 6 (Roche Molecular Biochemicals) for 3 hrs. The cells plus untreated cells as controls are rinsed once in ice-cold phosphate-buffered saline and grown at 37° C., 5% CO₂ in the appropriate medium for 16 hrs. The cells are lysed in Lysis Reagent (Promega) following the manufacturer's instructions. FIG. 14 illustrates the transfection schemes. The diagram shows the transient transfection of DNAs for p38MAPK, PTP, STAT6(B) and LITAF. The series of STAT6(B)* constructs includes wild-type and several serial mutations. The series of LITAF** constructs includes wildtype and several serial mutations. These constructs are transiently transfected into macrophages consistent with the Figure.

Kinases or Phosphatases Involvement in the Interaction Between LITAF and STAT6(B) by Immunoprecipitation and EMSA.

Immunoprecipitation and EMSA are used to investigate whether phosphorylation, as tested via overexpression of p38 MAPK, significantly affects the nuclear translocation and LITAF and STAT6(B) protein levels in nucleus. Comparison of treated cells (lanes 7, 9 and 12 in FIG. 14) with control cells provide definitive answers. For the dephosphorylation, similar approach is used and PTP over expressed, to test whether it specifically affects the nuclear translocation and decreases LITAF and STAT6(B) levels. Comparison of treated cells (lane 10, 11 and 13 in FIG. 14) with control cells provide definitive answers.

a. Immunoprecipitation: Mouse macrophages are transiently transfected with DNAs (FIG. 14). Both the treated cells and untreated control cells are grown in RPMI 1640 medium with 10% FCS. After incubation, cells are rinsed once in ice-cold phosphate-buffered saline and lysed in Lysis Reagent (Promega) following the manufacturer's instructions. The cell lysates extracted from whole cell and nucleus described above are respectively immunoprecipitated with 2 μg of antibody, either Stat6 (S-20, Santa Cruz) or LITAF (611615, BD Biosciences) for 2 hrs at 4° C., followed by incubation with 20 μl of protein A/G plus-Agarose-Sepharose beads (SC-2003, Santa Cruz) for an additional 1 h. The beads are washed three times in PBS buffer and then suspended in SDS sample buffer heated to 95° C. for 5 min. The eluted proteins are applied to SDS-polyacrylamide gels and proteins are detected by Western blotting with antibodies: Stat6, LITAF, Actin (C-11, Santa Cruz) and tubulin (C-20, Santa Cruz) as control.

b. EMSA: To confirm binding, EMSA is performed as follows: a reaction mixture containing 1 μg of lysate from the treated cells as described (Table 7, lane 4 or 5) plus 0.1 μg GST-LITAF and/or 0.1 μg GST-STAT6(B) fusion proteins purified following the manufacturer's instructions (Pharmacia), 1 μl radiolabeled (1×10⁵ cp/μl, 2 pmol) double stranded oligonucleotide TNF-promoter DNA including a sequence, CTCCC (the specific binding site, −515˜−511, to LITAF (see, Tang, et al., PNAS USA, 100:4096-4101, 2003), 3 μg poly(dI/dC), 5 μg bovine serum albumin, 4 μl gel shift binding 5× buffer (Promega), and nuclease-free water to 20 μl, is incubated at room temperature for 30 min prior to electrophoresis on non-denaturing 6% polyacrylamide gels in Tris-borate-EDTA buffer (90 mM Tris-borate, 2 mM EDTA HEPES [pH8.0]) followed by autoradiographic exposure of film. Finally, the supershifted protein bands are analyzed to confirm that STAT6(B) or/and LITAF is further bound with p38MAP or PTP.

In vitro kinase and phosphatase assays. In vitro kinase assays are performed to confirm that p38MAPK phosphorylates STAT6(B) and/or LITAF. 1 μg GST fusion protein of STAT6(B) as described above is incubated at room temperature for 1 h with 0.5 U/μl of cPKA (Invitrogen) in the presence of 2 μCi of γ-[³²P]ATP, 10 μM ATP and 8 mM MgCl₂ (20 μl final volume). The reactions is stopped by adding SDS sample buffer and boiling followed by SDS-PAGE and autoradiography as described (Slack, et al., J Biol Chem, 276:16491-16500, 2001; Korporaal, et al., J Biol Chem, 279:52526-52534, 2004). In vitro phosphatase assays will be performed to confirm that PTP dephosphorylates STAT6(B) and/or LITAF with a Universal Tyrosine Phosphatase Assay Kit (Cat# MK411, Takara Inc, Tokyo, Japan) following the manufacturer's instructions. These assays will help to confirm that 1) p38MAP kinase and/or PTP are involved in the phosphorylation/dephosphorylation of STAT6(B) and/or LITAF and 2) the sites that function as binding domains specific for phosphorylation/dephosphorylation.

ELISA. TNF ELISA is used as readout from treated cells to confirm that PTP prevents the interaction between STAT6(B) and LITAF, which would hamper translocation into the nucleus (as described in FIG. 14 lanes 1, 2, 5, 10, 11 and 13, with appropriate controls). Briefly, 1×10⁶ transiently transfected mouse macrophages are rinsed once in ice-cold phosphate-buffered saline and grown at 37° C., 5% CO₂ in the appropriate medium. After 24 hrs of incubation at −37° C., 5% CO₂, culture supernatants are harvested, centrifuged at 1,500×g to remove cell debris, then the secreted TNF is measured by ELISA (ABRAXIS, Hatboro, Pa.) and quantified on a Model 680 Microplate Reader (Biorad).

### Example 6

### This Prophetic Example Determines the Role(s) of LITAF and the —STAT6(B)-LITAF Complex in Regulating Proinflammatory Cytokine Gene Expression with Particular Emphasis on TNFα and MCP-1

The present Inventors have recently demonstrated that LITAF alone or together with STAT6(B) stimulates TNF-α and also other cytokines (i.e., MCP-1). The preliminary data showed that the increase in MCP-1 expression is transcriptionally regulated by LITAF and STAT6(B). To dissect the relevant mechanisms and determine whether they parallel the process that was observed for TNF-α, it is determined that the precise MCP-1 DNA binding site is involved in this process. Furthermore, muteins (modified proteins) of STAT6(B) or LITAF will allow the identification of the peptide sequence(s) required for DNA binding. This approach facilitates the design of drugs that disrupt this binding and inhibit deleterious expression of proinflammatory cytokines in inflammatory disease.

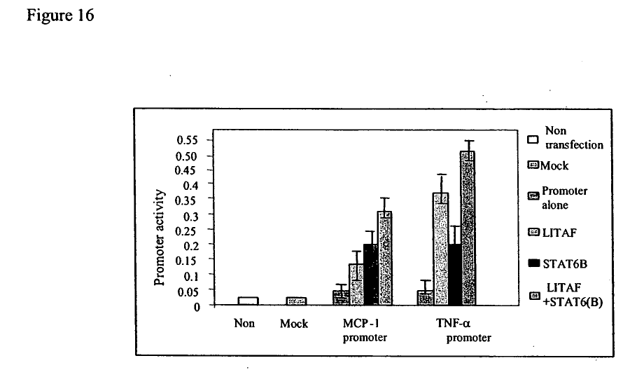

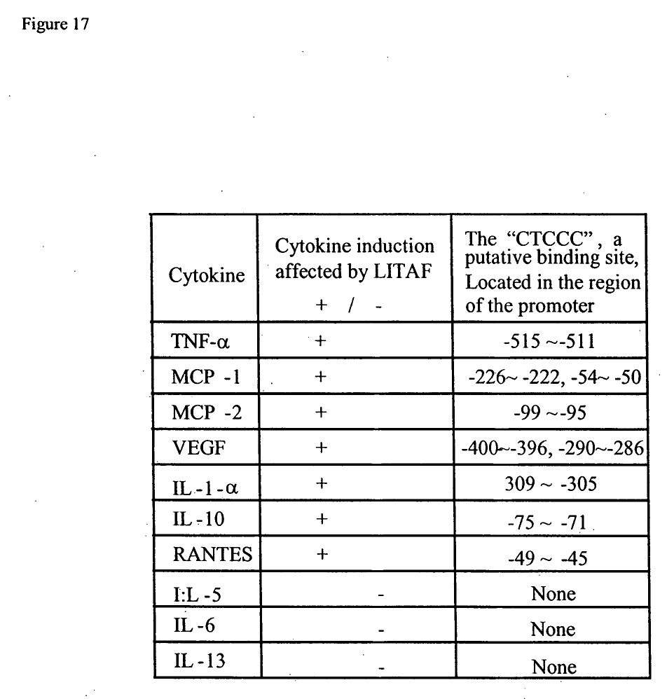

The data demonstrates that gene expression of proinflammatory cytokines other than TNFα is regulated by LITAF and STAT6(B) upon LPS stimulation. To show this hypothesis, monocyte chemoattractant protein-1 (MCP-1), an important component of the inflammatory process, is selected to determine whether its LPS-triggered transcriptional regulation parallels observations for TNF. Recent studies have shown that the MCP-1 promoter activity can be regulated by NF-KB, STAT1, CEB/P homologous protein (CHOP), heme oxygenase-1 (HO-1) or activated protein C (APC) (Kim, et al, Clin Exp Immunol, 140:450-460, 2005; Jaruga, et al., Am J Physiol Gastrointest Liver Physiol, 287:G471-479, 2004, Abraham, et al., J Neuroimmunol, 160:219-221, 2005; Kanakiriya, et al., Am J Physiol Renal Physiol, 284:F:546-554, 2003). However, the precise LPS signaling pathways leading to MCP-1 induction have not been fully identified. In Examples 1-3, above, the regulation of LPS-induced inflammatory cytokine expression was shown. The data showed that transient transfection of either LITAF alone or STAT6(B) alone significantly up-regulated MCP-1 promoter activity. Surprisingly, overexpression of both STAT6(B) and LITAF enhanced MCP-1 expression (see, FIG. 15a,b and FIG. 16). After comparing the MCP-1 promoter sequence with other STAT6(B)-LITAF complex-regulated cytokines, almost every-cytokine under this control displayed in its promoter a “CTCCC” sequence upstream of the TATA box (FIG. 17), the specific LITAF binding site identified on TNF-α promoter (Tang, et al., PNAS USA, 100:4096-4101, 2003). Thus, CTCCC represents a specific sequence in cytokine promoters to which LITAF binds for activity. In addition, it was demonstrated (see, Example 5, supra) that a peptide stretch on LITAF named peptide B was found to bind CTCCC and activate TNF promoter activity on its own (Tang, et al., PNAS USA; 100:4096-4101, 2003). Thus, short peptides with amino acids designed and synthesized based on the potential binding domain of LITAF and STAT6(B) jointly enhance MCP-1 promoter activity.

Thus, this Example will show that MCP-1 gene expression is, 1) transcriptionally regulated by LITAF via the MCP-1 “CTCCC” site and that this promoter activity may be enhanced by the addition of STAT6(B) and 2) follows the same pattern of activation as observed for TNFα. Furthermore, mutations of STAT6(B) and LITAF are used to isolate the peptide sequence(s) involved in protein-DNA binding. Consequently, these peptides including peptide B are used as stimuli for the regulation of MCP-1 promoter activity. This approach facilitates the design of pharmacological interventions aimed at reducing deleterious expression of proinflammatory cytokines in inflammatory disease.

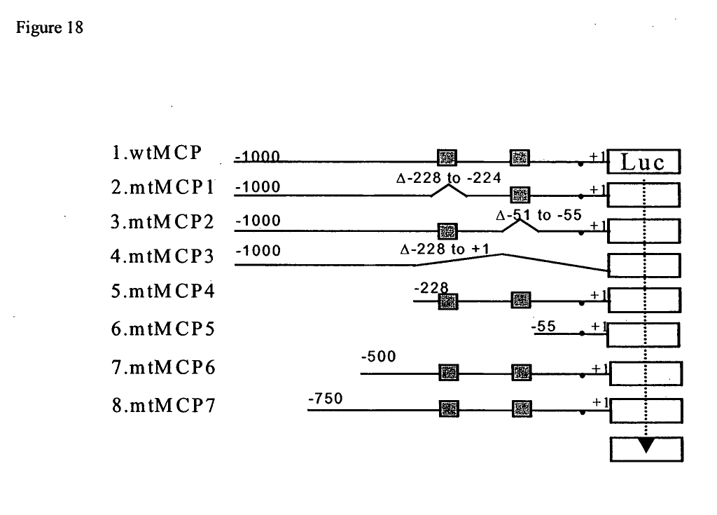

1. Plasmid construction. To confirm whether CTCCC in the MCP-1 promoter region is a specific sequence for LITAF binding activity, the following series of MCP-1 promoter DNA constructs (see, #1 to 4, FIG. 14) are prepared and subcloned into the pGL3-Basic vector (Pharmacia), a promoterless and enhancerless luciferase reporter gene. To further investigate an unknown sequence in the MCP-1 promoter for STAT6(B) binding activity or LITAF binding activity (if CTCCC is not specific) the series of MCP-1 promoter DNA fragments (see #1, 5 to 8, FIG. 18) are subcloned as shown in FIG. 18. The transcription initiation position in the MCP-1 promoter is indicated by a small solid dot. The DNA sequence encompassing 1,000 bp upstream from the initiation position is considered as full length (No. 1) and the other deletions (No. 2-8) will be generated by PCR with the appropriate primers, and inserted into a pGL3basic plasmid. The CTCCC sequence locations are indicated by small gray squares.

(1) wtMCP (−1000 to +1) is generated by PCR with primer pairs: 5′-aatgtttctagattctcctttagc-3′ [SEQ ID NO.: 29] and 5′-cctctggttcctctggctgctgt-3′ [SEQ ID NO.: 30]. (2) mtMCP1 (−1000 to 1Δ-228 to -224). The first in-frame mutant was generated by PCR with primer pairs, 5′-aatgtttctagattctcctttagc-3′ [SEQ ID NO.: 31] and 5′-gatggggtgccattaagcccagactgaccacaga-3′ [SEQ ID NO.: 32]. The second mutant DNA was generated by PCR with primer pairs, 5′-gcaccccatccamgctcatttggtc-3′ [SEQ ID NO.: 33] and 5′-ctctcggttcctctggctgctgt-3′ [SEQ ID NO.: 34]. Both first and second DNA fragments above were purified and diluted as template to 1 ng/reaction and finally amplified by PCR with primer pairs: 5′-aatgtttctagattetcctttagc-3′ [SEQ ID NO.: 35] and 5′-cctctcggttcctctggctgctgt-3′ [SEQ ID NO.: 36]. (3) mtMCP2 (−1000 to 1Δ-55 to -51). The first in-frame mutant was generated by PCR with primer pairs, 5′-aatgtttctagattctcctttagc-3′ [SEQ ID NO.: 37] and 5′-agagggcggagtcaagcaggaggagggatct-3′ [SEQ ID NO.: 38]. The second mutant DNA was generated by PCR with primer pairs, 5′-ctccgccctcttctgcccgctttcaataagag-3′ [SEQ ID NO.: 39] and 5′-cctctcggttectctggctgctgt-3′ [SEQ ID NO.: 40]. Both first and second DNA fragments above were purified and diluted as template to 1 ng/reaction and finally amplified by PCR with primer pairs, 5′-aatgtttctagattctcctttagc-3′ [SEQ ID NO.: 41] and 5′-cctctcggttcctctggctgctgt-3′ [SEQ ID NO.: 42]. (4) mtMCP3 (−1000 to −229) was generated by annealing with primer pairs: 5′-aatgtttctagattctcctttagc-3′ [SEQ ID NO.: 43] and 5′-gatggggtgccattaagcccagactgaccacaga-3′ [SEQ ID NO.: 44]. (5) mCMCP4 (−228 to +1) was generated by annealing with primer pairs: 5′-gcaccccatccattgctcatttggtc-3′ [SEQ ID NO.: 45] and 5′-cctctcggttcctctggctgctgt-3′ [SEQ ID NO.: 46]. (6) mtMCP5 (−55 to +1) was generated by annealing with primer pairs: 5′-gcacccatccatttgctcatttggtc-3′ [SEQ ID NO.: 47] and 5′-cctctcggttcctctggctgctgt-3′ [SEQ ID NO.: 48]. (7) mtMCP6 (−500 to +1) was generated by annealing with primer pairs: 5′-geaccccatccatttgctcatttggtc-3′ [SEQ ID NO.: 49] and 5′-cctctcggttcctctggctgctgt-3′ [SEQ ID NO.: 50]. (8) mtMCP7 (−750 to +1) was generated by annealing with primer pairs: 5′-gcaccccatccatttgctcatttggtc-3′ [SEQ ID NO.: 51] and 5′-cctctcggttcctctggctgctgt-3′ [SEQ ID NO.: 52]. (9) pCDNAHA-STAT6(B), pCDNAHA, wtTNFP and mtTNFP1 used for this test are available from our lab. (10) Additional point or site mutations of DNA constructs that alter their specificity for binding activity are identified at the next step, and these are made as needed.

2. Transient Transfection and Luciferase Assay. To determine whether the MCP-1 promoter contains LITAF and/or STAT6(B) binding activity, luciferase assays are performed. 1×10⁶ U2OS cells (Human Tibia Sarcoma Cells amenable to high transfection efficiency) are co-transfected with 1 μg DNAs including the series of MCP-1 promoter/reporter constructs or wtTNFP and mtTNFP1 as controls, and pCDNAHA-STAT6(B) or pCDNAHA-LITAF by Fugene 6 (Roche Molecular Biochemicals) and will be grown at 37° C., 5% CO₂ in the appropriate medium for 16 hrs. The β-galactosidase gene is included in all transfections. Cells are lysed in Lysis Reagent (Promega) following the manufacturer's instructions. The luciferase activity of each lysate is measured (Turner Designs luminometer model TD-20/20) using a commercial kit (Luciferase Reporter Assay System, Promega) according to the protocol provided by the manufacturer. Finally, the data is normalized to β-galactosidase expression.

3. DNase I Footprinting. To determine the exact DNA sequence involved in LITAF and STAT6(B) binding of the MCP-1 promoter, the DNase I footprinting method is used as described (Tang, et al., PNAS USA, 100:4096-4101, 2003). The GST-STAT6(B) or GST-LITAF as described above (Example 5, above) is used for this test. The putative oligonucleotide identified in the MCP-1 promoter for binding activity to be confirmed by luciferase assay (above) is designed and synthesized. The oligo DNA (0.5 μg) as probe is labeled with γ-[³²P]ATP by T4 polynucleotide kinase (Promega) as described Tang, et al., PNAS USA, 100:4096-4101, 2003). Labeled DNA is then purified using a G-25 Sephadex column (Boehringer) and precipitated with ethanol. After centrifugation, the DNA pellet is suspended in 10 μl water. The γ-[³²P]ATP-labeled DNA (1×10⁵ cpm/μl, 2 pmol) is then mixed with 25 μl binding buffer (Promega), 0.1 μl GST-hLITAF or STAT6(B) fusion protein (GST fusion protein alone as control), and nuclease-free water (Promega) to 50 μl, and incubated on ice for 30 min. Then, 50 μl of Ca²/Mg² solution at room temperature is added to the reaction mixture and incubated for one minute, followed by addition of 3 μl DNase I (Promega) gentle mixing, incubation for an additional 5 min and, finally, reaction termination. The reaction mixture is treated with phenol and precipitated with ethanol. After centrifugation, the DNA pellet is suspended in 5 μl of TE buffer. The sample is applied to a 6% polyacrylamide sequencing gel (Invitrogen) followed by autoradiographic exposure of film. Finally, data analysis as described above identifies putative protein-DNA binding domain.

4. EMSA. In order to identify the interaction of STAT6(B)-LITAF complex with MCP-1 promoter, EMSA is performed. A reaction mixture containing 0.1 μg GST-LITAF and/or 0.1 μg GST-STAT6(B) fusion proteins, 1 μl γ-[³²P]ATP-labeled (1×10⁵ cpm/μl, 2 pmol) double stranded oligonucleotide MCP-1 promoter DNA (identified in above), 3 μg poly(dI/dC), 5 μg bovine serum albumin, 4 μl gel shift binding 5× buffer (Promega), and nuclease-free water to 20 μl, is incubated at room temperature for 30 min prior to electrophoresis on non-denaturing 6% polyacrylamide gels in Tris-borate-EDTA buffer (90 mM Tris-borate, 2 mM EDTA HEPES [pH 8.0]) followed by autoradiographic exposure of film. The analysis of this data should definitively identify the protein-DNA binding domain.

5. Peptide design, synthesis and testing. Based on the specific binding domain of STAT6(B) or LITAF (as confirmed above) peptides are designed and synthesized (including LITAF peptide B) by site mutations of STAT6(B) and LITAF. These peptides are then used as stimuli for the regulation of exogenous (a) and endogenous (b) MCP-1 promoter activity. Hemagglutinin antigenic peptide (HA) as control consists of the sequence (YPYDVPDY ASL [SEQ ID NO: 53]). All peptides are solubilized in DMSO.

(a) 1×10⁶ U2OS cells are co-transfected with 1 μg DNAs including the series of MCP-1 promoter/reporter constructs or wtTNFP and mtTNFP1 as control, by Fugene 6 (Roche Molecular Biochemicals) for 3 hrs. Cells are washed with PBS and then cells in the appropriate media are treated with peptides or without peptides as a control, using a Chariot kit that is a new transfection reagent capable of efficiently delivering proteins, peptides and antibodies into cultured mammalian cells in less than 2 hrs (Chariot Motif, Carlsbad, Calif.) and incubated overnight at 37° C., 5% CO₂. The β-galactosidase gene is included in all transfections. Cells are lysed in Lysis Reagent (Promega) following the manufacturer's instructions. The luciferase activity of each lysate is measured (Turner Designs luminometer model TD-20/20) using a commercial kit (Luciferase Reporter Assay System, Promega) according to the protocol provided by the manufacturer. Finally, the data is normalized to β-galactosidase expression and analyzed as described above.

(b) After pretreatment with PMA, THP-1 cells are stimulated with 1 μg/ml peptides or without peptides as control, by Chariot kit (Chariot) for 24 hrs. The culture supernatants are harvested and MCP-1 or TNF-α as control is quantified by ELISA. HA peptide is used as a negative control.

6. ELISA. To quantify MCP-1 production (TNFα as control) ELISA is performed. THP-1 cells are induced to maturation by addition of 200 mM PMA (Sigma) and incubated at 37° C., 5% CO₂ for 20 hrs, then washed with PBS twice and stimulated with the Chariot/peptide complex in a 96-well plate at 2×10⁴ cells/well as indicated (Tang, et al., PNAS USA, 100:4096-4101, 2003). After 24 hrs of incubation at 37° C., 5% CO₂, culture supernatants are harvested, centrifuged at 1,500×g for 5 min to remove cell debris, and the secreted protein is measured by ELISA (R&D System, Minneapolis, Minn.) and quantified on a Model 680 Microplate Reader (Biorad).

The Example shows the exact DNA binding sequence involved in MCP-1 promoter activation (#1-3, 9 and 10, FIG. 18). In addition, this Example determines STAT6(B) and LITAF amino acid sequences involved in the DNA binding, and demonstrates the design and synthesis of the peptides accordingly. These peptides are used to reproduce our data obtained with native protein. Overall, this data will provide a better understanding of the mechanism involved in LITAF-STAT6(B) complex activation of cytokine gene expression. The successful targeting of LITAF and STAT6(B) activity using blocking agents will be valuable in strategies for reducing deleterious expression of proinflammatory cytokines in inflammatory diseases. Target approaches have already started to screen available libraries (FDA 2000; Nutraceuticals; Harvard Library) for potential hits targeting LITAF activities. The use of blocking antibodies as was done with for IL-1 and TNF (Assuma, et al., J Immunol, 160:403-409, 1998) may be other alternatives.

### Example 7

### This Prophetic Example Determines the Involvement of STAT6(B) Alone or STAT6(B)-LITAF Complex in the Regulation of VEGF Gene Expression

As shown in Example 4 STAT6(B) alone stimulates VEGF expression while the LITAF-STAT6(B) complex inhibits it. These data show STAT6(B) transcriptional regulation of VEGF. To dissect this mechanism, VEGF promoter constructs are generated to determine the exact DNA binding site involved in this transcriptional regulation. In addition, muteins of STAT6(B) are used to isolate the peptide sequence(s) involved in protein-DNA binding. Furthermore the inhibitory effect of the complex is tested by the same approach. This strategy facilitates the design of drugs aimed at modulating VEGF expression in angiogenesis.

Vascular endothelial growth factor (VEGF) is a potent angiogenic factor (Mukhopadhyay, et al., Semin Cancer Biol, 14:123-130, 2004). In vivo studies have shown that suppression of VEGF inhibits tumor growth and anti-VEGF antibodies or VEGF inhibitors also decrease the metastasis of human tumors implanted into nude mice (Kuba, et al., Cancer Res, 60:6737-6743, 2000). Therefore, mechanisms involved in the regulation of the VEGF promoter activity have been widely studied (Abid, et al., J Biol Chem, 279:44030-44038, 2004; Akiyama, et al., Am J Physiol Cell Physiol, 288:C913-920, 2005; Finkenzeller, et al., Angiogenesis, 7:59-68, 2004). A recent study has shown that the IL/JAK/STAT3/NFkB pathway is involved in the regulation of VEGF promoter activity (Gong, et al., Clin Cancer Res, 11: 1386-1393, 2005). The data in Example 4 have shown that transient transfection of STAT6(B) alone in THP-1 cells significantly upregulates VEGF promoter activity. Contrarily, co-transfection of both STAT6(B) and LITAF reduces VEGF gene transcription (FIG. 15b and FIG. 11). Furthermore, STAT6(B)-transfected TBP1 cell supernatant stimulated in vitro angiogenesis while supernatant from THP1 cells co-transfected with both LITAF and STAT6(B) did not. Finally, analysis of the VEGF promoter sequence shows that it contains a “CTCCC” in two locations (FIG. 17), a motif that was a DNA binding site specific to LITAF in the TNF-promoter (Tang, et al., PNAS USA, 100:4096-4101, 2003). The data present here show a well characterized regulating system capable of tuning VEGF expression on or off. STAT6(B) upregulates VEGF gene transcription by binding a specific site on the VEGF promoter, and that this binding is inhibited when LITAF is also present. Therefore, LITAF plays a key role in this regulatory switch.

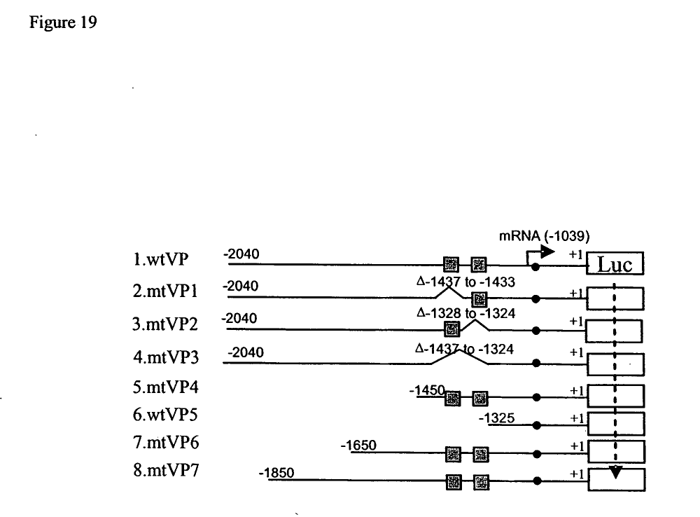

The regulation of VEGF expression is poorly understood and, therefore, warrants a more comprehensive analysis. The following experiments in this Example: [1] identify a STAT6(B) binding domain in the VEGF promoter, [2] determine whether LITAF interacts with CTCCC or other specific site(s); and [3] characterize VEGF promoter activity in the presence or absence of these specific sites. Mutations of STAT6(B) or LITAF are used to identify the peptide sequence involved in protein-DNA binding. This approach facilitates the design of pharmacological interventions aimed at controlling VEGF expression in angiogenesis. A diagram of VEGF promoter constructs is shown in FIG. 19. The transcription initiation position at −1039 in VEGF promoter is indicated by a small solid dot. The DNA sequence encompassing 1,000 bp upstream from this position is considered full length (No. 1) and the other deletions (No. 2˜8) will be generated by PCR with the appropriate primers, and inserted into a pGL3basic plasmid. The CTCCC sequence is indicated by a small gray square.

1. Plasmid construction. To determine the sequence in the VEGF promoter for STAT6(B) binding, a series of VEGF promoter DNA constructs (above) are necessary and the mutation constructs are used for several assays below. Since the complex LITAF-STAT6(B) inhibits VEGF while LITAF alone does not have any effect, it is possible that LITAF binds to VEGF promoter and occupies a DNA site. To determine whether CTCCC is the site on the VEGF promoter for LITAF binding activity the following series of VEGF promoter DNA constructs (above) are required and subcloned into the pGL3-Basic vector (Pharmacia).

(1) wtVP (−2040 to +1) will be generated by PCR with primer pairs: 5′-atctggggttgggggggcagca-3′ [SEQ ID NO.: 54] and 5′-catggtttcggaggeccgacc-3 [SEQ ID NO.: 55]. (2) mtVP1 (−2040 to +1Δ-1437 to -1433). The first in-frame mutant will be generated by PCR with primer pairs, 5′-atctggggttgggggggcagca-3′ [SEQ ID NO.: and 5′-agggacacacagatctgttgg-3′ [SEQ ID NO.: 57]. The second mutant DNA will be generated by PCR with primer pairs, 5′-ctgtgtgtccctcacccgtccctgtccggctct-3′ (SEQ ID NO.: 58] and 5′-catggtttcggaggcccgacc-3′ [SEQ ID NO.: 59]. Both first and second DNA fragments above will be purified and diluted as template to 1 ng/reaction and finally amplified by PCR with primer pairs: 5′-atctggggttgggggggcagca-3′ [SEQ ID NO.: 60] and 5′-catggtttcggaggcccgacc-3′ [SEQ ID NO.: 61]. (3) mtVP2 (−2040 to +1Δ-1328 to -1324). The first in-frame mutant will be generated by PCR with primer pairs, 5′-atctggggttgggggggcagca-3′ [SEQ ID NO.: 62] and 5′-caggaaagtgaggttacgtgc-3′ [SEQ ID NO.: 63]. The second mutant DNA will be generated by PCR with primer pairs, 5′-tcactttcctgtcctcgccaatgccccgcggg-3′ [SEQ ID NO.: 64] and 5′-catggtttcggaggcccgacc-3′ [SEQ ID NO.: 65]. Both first and second DNA fragments above will be purified and diluted as template to 1 ng/reaction and finally amplified by PCR with primer pairs, 5′-atctggggttgggggggcagca-3′ [SEQ ID NO.: 66] and 5′-catggtttcggaggcccgacc-3′ [SEQ ID NO.: 67]. (4) mtVP3 (−2040 to +1Δ-1437 to -1324) The first in-frame mutant will be generated by PCR with primer pairs, 5′-atctggggtfgggggggcagca-3′ [SEQ ID NO.: 68] and 5′-agggacacacagatctgttgg-3′ [SEQ ID NO.: 69]. The second mutant DNA will be generated by PCR with primer pairs, 5′-ctgtgtgtcccttcctcgccaatgcccgcggg-3′ [SEQ ID NO.: 70] and 5′-catggtttcggaggcccgacc-3′ [SEQ ID NO.: 71]. Both first and second DNA fragments above will be purified and diluted as template to 1 ng/reaction and finally amplified by PCR with primer pairs, 5′-atctggggttgggggggcagca-3′ [SEQ ID NO.: 72] and 5′-catggtttcggaggcccgacc-3′ [SEQ ID NO.: 73]. (5) mtVP4 (−1450 to +1) will be generated by annealing with primer pairs: 5′-tctgtgtgtccctctccccac-3′ [SEQ ID NO.: 74] and 5′-catggtttcggaggcccgacc-3′ [SEQ ID NO.: 75]. (6) mtVP5 (−1325 to +1) will be generated by annealing with primer pairs: 5′-tcctcgccaatgccccgcgg-3′ [SEQ ID NO.: 76] and 5′-catggtttcggaggcccgacc-3′ [SEQ ID NO.: 77]. (7) mtVP6 (−1650 to +1) will be generated by annealing with primer pairs: 5′-ctctttagccagagccggggt-3′ [SEQ ID NO.: 78] and 5′-catggtttcggaggcccgacc-3′ [SEQ ID NO.: 79]. (8) mtVP7 (−1850 to +1) will be generated by annealing with primer pairs: 5′-tgccgctcactttgatgtctg-3′ [SEQ ID NO.: 80] and 5′-catggtttcggaggcccgacc-3′ [SEQ ID NO.: 81]. (9) The expression plasmids pCDNAHA-STAT6(B), pCDNAHA, wtTNFP and mtTNFP1 is used for this test are readily available or easily made by one skilled in the art (10) If additional-point or site mutations of DNA constructs that alter their specificity for binding activity are needed at the next step they and will be made.

Transient Transfection and Luciferase Assays. To determine whether the VEGF promoter contains LITAF and or STAT6(B) binding activity the luciferase assay is performed. 1×10⁶ U2OS cells are co-transfected with 1 μg DNAs including the series of VEGF promoter/reporter constructs or wtTNFP and mtTNFP1 as controls and pCDNAHA-STAT6(B) or pCDNAHA-LITAF by Fugene 6 (Roche Molecular Biochemicals) and are grown at 37° C., 5% CO₂ in the appropriate medium for 16 hrs. The β-galactosidase gene will be included in all transfections. Cells will be lysed in Lysis Reagent (Promega) following the manufacturer's instructions. The luciferase activity of each lysate will be measured (Turner Designs luminometer model TD-20120) using a commercial kit (Luciferase Reporter Assay System, Promega) according to the protocol provided by the manufacturer. Finally, the data is normalized to β-galactosidase expression.

3. DNase I Footprinting. To determine the minimum DNA sequence involved in LITAF and STAT6(B) binding sites in the VEGF promoter, DNase I footprinting method as previously described and in (Tang, et al., PNAS USA, 100:4096-4101, 2003) is used. The GST-STAT6(B) or GST-LITAF as described Example 5 is used for this test. The putative oligonucleotide to be identified in the VEGF promoter for binding activity is confirmed by luciferase assay (above) then designed and synthesized. The GST-STAT6(B) or GST-LITAF fusion proteins described above Example 5 are ready for this test. The oligo DNA (0.5 μg) as probe is labeled with [γ-³²P]ATP by T4 polynucleotide kinase (Promega) as described (Tang, et al., PNAS USA, 100:4096-4101, 2003). Labeled DNA is purified using a G-25 Sephadex column (Boehringer) and precipitated with ethanol. After centrifugation, the DNA pellet is suspended in 10 μl water. The γ-[³²P]ATP-labeled DNA (1×10⁵ cp/μl, 2 pmol) is then mixed with 25 μl binding buffer (Promega), 0.1 μl GST-hLITAF or STAT6(B) fusion protein (GST fusion protein alone as control), and nuclease-free water (Promega) to 50 μl, incubated on ice for 30 min, to which 50 μl Ca²/Mg² solution at room temperature is added and incubated for one min, then 3 μl DNase I (Promega) is added, mixed gently, then incubated for an additional 5 min, followed by reaction termination. The reaction mixture is treated with phenol and precipitated with ethanol. After centrifugation, the DNA pellet is suspended in 5 μl of TE buffer. The sample is applied to a 6% polyacrylamide sequencing gel (Invitrogen) followed by autoradiographic exposure of film. Finally, the data analysis helps to identify a protein-DNA binding domain.

4. EMSA. In order to identify the interaction of STAT6(B)-LITAF complex with VEGF promoter, EMSA is used. A reaction mixture containing 0.1 μg GST-LITAF and/or 0.1 μg GST-STAT6(B) fusion proteins, 1 μl γ-[³²P]ATP-labeled (1×10⁵ cpm/μl, 2 pmol) double stranded oligonucleotide VEGF promoter DNA, 3 μg poly(dI/dC), 5 μg bovine serum albumin, 4 μl gel shift binding 5× buffer (Promega), and nuclease-free water to 20 μl, is incubated at room temperature for 30 min prior to electrophoresis on non-denaturing 6% polyacrylamide gels in Tris-borate-EDTA buffer (90 mM Tris-borate, 2 mM EDTA HEPES [pH18.0]) followed by autoradiographic exposure of film. Finally, data analysis helps to identify a protein-DNA binding domain.

5. Peptides design, synthesis and testing. Based on the specific binding domain of STAT6(B) or LITAF (as confirmed above), peptides are designed and synthesized (including LITAF peptide B) by site mutations of STAT6(B) and LITAF. These peptides are then used as stimuli for the regulation of exogenous (a) or endogenous (b) VEGF promoter activity. Hemagglutin antigenic peptide (HA) is used as control. All peptides are solubilized in DMSO.

(a) 1×10⁶ U2OS cells are co-transfected with 1 μg DNAs including the series of VEGF promoter/reporter constructs or wtTNFP and mtTNFP1 as control by Fugene 6 (Roche Molecular Biochemicals) for 3 hrs. Cells are washed with PBS, and then cells in the appropriate media are treated with peptides, or without peptide as control, by Chariot kit (Chariot) overnight at 37° C., 5% CO₂. The β-galactosidase gene ia included in all transfections. Cells are lysed in Lysis Reagent (Promega) following the manufacturer's instructions. The luciferase activity of each lysate is measured (Turner Designs luminometer model TD-20/20) using a commercial kit (Luciferase Reporter Assay System, Promega) according to the protocol provided by the manufacturer. Finally, the data ia normalized to β-galactosidase expression and analyzed as described (above).

(b) After pretreatment with PMA, THP-1 cells are then stimulated with 1 μg/ml peptides or without peptides as control by Chariot kit (Chariot) for 24 hrs. The culture supernatants are harvested and VEGF or TNF-α as control are quantified by ELISA. The HA-peptide is used as negative control.

6. ELISA. THP-1 cells are induced to maturation by addition of 200 nM PMA (Sigma) and incubated at 37° C., 5% CO₂ for 20 hrs, then washed with PBS twice, and stimulated-(delivered) with Chariot/peptide complex of peptide in a 96-well plate at 2×10⁴ cells/well as described (Tang, et al., PNAS USA, 100:4096-4101, 2003). After 24 hrs of incubation at 37° C., 5% CO₂, culture supernatants are harvested, centrifuged at 1,500×g for 5 min to remove cell debris, and then the concentration of secreted protein is measured by ELISA (R&D System, Minneapolis, Minn.) and quantified on a Model 680 Microplate Reader (Biorad).

In this Example the minimum STAT6(B) DNA binding sequence to activate VEGF promoter is identified. In addition, these experiments determine if “CTCCC” is the LITAF sequence for the down-regulation of VEGF. Should “CTCCC” turn out not to be the sequence, we will identify the sequence in the VEGF promoter for LITAF binding activity with the other mutations as described in (FIG. 19 #1-4, 9 and 10 and above). In this case, mutations spanning shorter sequences are then required. In addition, STAT6(B) and LITAF amino acid sequences involved in the DNA binding are determined and peptides are designed and synthesized accordingly. These peptides are used to validate our data obtained with native proteins. Overall, these data provide a better understanding of the mechanisms involved in STAT6(B) activation and STAT6(B)-LITAF complex inhibition of VEGF expression. Ultimately the results of these experiments will serve to design strategies aimed at promoting or reducing angiogenesis.

### Example 8

### This Prophetic Example Compares the Involvement of LITAF Alone Vs. STAT6(B)-LITAF Complex, as Well as STAT6(B) Alone vs. STAT6(B)LITAF Complex in Animal Models of Inflammation and Angiogenesis

We have now produced LITAF-deficient mice (see, Example 2, supra). To evaluate STAT6(B)'s specific role in vivo STAT6(B)-deficient animals will be generated as follows. For testing the enhanced effect of the complex in inflammation, LPS-induced processes in LITAF−/− animals vs. animals lacking both LITAF and STAT6(B) are compared. For testing the stimulatory effect of STAT6(B) on VEGF and the inhibitory effect of the complex, angiogenic processes in STAT6(B)−/− vs. doubly deficient LITAF-STAT6(B) animals are compared. The data from these animal model experiments are used to substantiate the in vitro observations of the Examples, above, and will enable the use of pharmacotherapies.

1. Generation of STAT6(B)-deficient mice. a. Cloning of mouse STAT6(B) cDNA and genomic DNA. Given the high expression of STAT6(B) in spleen, a mouse STAT6(B) probe is used to screen a mouse spleen cDNA library (Clontech). STAT6(B) cDNA is used as a probe to screen murine STAT6(B) genomic DNA (whole gene) from the mouse BAC 129/Svj library (Bolcato-Bellemin, et al., J Endotoxin Res, 10:15-23, 2004) and the genomic clone is sequenced and fully mapped. Finally, chromosomal localization is determined by FISH and its physical map measured using restriction enzymes. A targeting vector spanning critical exons is designed.

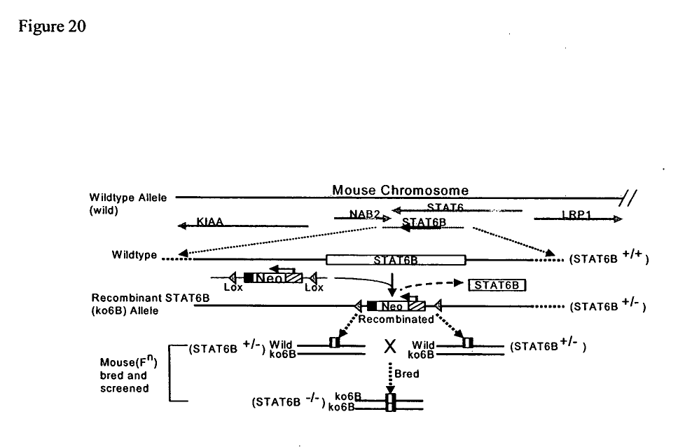

b. Mapping of the murine STAT6(B) locus and design of targeting vector. The genomic DNA of the whole murine STAT6(B) gene is mapped based on the results obtained above. A targeted mutation is designed using a kit (Constitutive Knock-out, Genoway). For the targeting vector (purchased from Genoway) a genomic fragment containing STAT6(B) is replaced by a neomycin resistance gene. The predicted structure of the STAT6(B) gene after targeted integration of the vector shown in FIG. 20 as the recombinant allele. The map of the targeted mutation is shown in FIG. 20. Note that two LoxP sites flanking the neo cassette are inserted. c. Generation of the knock out mice (STAT6(B)^{-/-}) and the double knock out mice (STAT6(B)^{-/-}, LITAF^{-/-}). After homologous recombination between STAT6(B) and neomycin in Embryonic Stem cells, the blastocyst injection is performed. Once chimeras are obtained, the animals are bred and backcrossed to the F6 generation before being tested, as above to create the LITAF-deficient animals. FIG. 20 shows a diagram of the generation of STAT6(B) knock-out mice. The mouse chromosome map shown here is referred to human chromosome map (NC_{—}000112, Genbank). Once the STAT6(B)-deficient animals have been generated, they are bred with the LITAF-deficient animals, to obtain LITAF-STAT6(B) doubly deficient animals. These animals are screened by real time PCR using specific PCR primers.

2. In vivo testing of Inflammation. a. Compare responses to systemic LPS challenge in LITAF−/−, LITAF-STAT6(B)−/− and wild-type animals in vivo. LITAF as well as STAT6(B) gene products significantly influence LPS-induced lethality. Sepsis and septic shock are systemic conditions that exhibit a high mortality rate. Endogenous mediators such as TNF, IL-1, IL-6 and IL-12 released in response to LPS have been identified as principal mediators of for example, pathology in sepsis, periodontal disease (Beutler, et al., Science, 229:869-871, 1985; Bazzoni and Beutler, J Inflamm, 45:221-238, 1995; Bone, Ann. Inter Med, 115:457-469, 1991). In murine models it is well accepted that D-galactosamine (D-GalN) dramatically sensitizes mice to the lethal effects of LPS via toxic effects on hepatocytes (Galanos, et al., PNAS USA, 76:5939-5943, 1979). There is agreement that death in LPS/D-GalN-challenged animals is due to TNF toxicity (Ashenazi, et al. PNAS USA, 88:10535-10539, 1991; Freudenberg, et al., Immun Infekt, 21:40-44, 1993). D-GalN-sensitized wild-type mice are sensitive to the lethal effect of LPS, even at a dose 100 fold less than the lethal dose for unsensitized littermates. The levels of LPS required to induce death in the D-GalN-sensitized wild-type mice are more similar to the human than the amount of LPS that would be required to induce the same level of lethality in unsensitized mice. Therefore, this model is used in LITAF-STAT6(B) doubly deficient, LITAF-deficient mice and in corresponding wild-type animals to assess the importance of the complex STAT6(B)-LITAF in LPS-induced processes compared to LITAF alone. Thus, using unsensitized mice and higher doses of LPS, is a novel model of sepsis in which other cytokines (i.e. in addition to TNF) significantly contribute to LPS toxicity. Furthermore, the D-GalN model allows us to evaluate the response of LITAF-STAT6(B)−/− mice compared to LITAF−/− mice to LPS challenge under conditions where lethality is exclusively determined by the production of TNF and not other cytokines.

Experimental approach. To assess the involvement of LITAF and STAT6(B) in LPS-induced cytokine production, the animals (LITAF−/−, double LITAF-STAT6(B)−/−, wild type mice) with D-GalN are sensitized and subsequently challenged systemically with various amounts of LPS (0.001-1 μg). For each of these experimental conditions, serum cytokines released into the circulation are assayed and profiled over a three-hour time course. TNF, IL-1, IL-6 and IL-12 levels each are assayed in mutant and wild type mice under the LPS challenge conditions described above.

c. Lethal dose of LPS with and without D-Gal sensitization. Dose-response curves for a series of challenges with P. gingivalis LPS, and then assess the LD₅₀ are compared. For D-GalN sensitized mice, a dose range of 0.001 to 0.1000 μg/50 ul of P. gingivalis LPS is used (20 g/mouse); For unsensitized mice a range of 100-1000 μg P. gingivalis LPS ia used. For statistical significance, 6 mice are used for each experimental point, based on a power analysis and previous published results (Li et al., Infect Immun, 70:3915-3922, 2002; Li, et al., Circulation, 105:861-867, 2002). A shift of the dose-response curve to the right or to the left indicates lesser or greater sensitivity, respectively, to LPS, compared with wild-type mice, whereas overlapping curves indicate equivalent sensitivities.

d. Kinetics of cytokine production. Administration of P. gingivalis LPS to mice is known to induce their macrophages to secrete a variety of proinflammatory cytolines, including TNF, IL-1 IL-6, and IL-12 as we have shown recently (Zhou, et al., Infect Immun, 73:935-943, 2005). The kinetics of systemic TNF, IL-1, IL-6 and IL-12 release in LITAF−/− and wild type litter mates using sublethal doses of LPS are measured. These experiments provide valuable insights into the role that the complex LITAF-STAT6 plays in the production of other cytokines following LPS challenge compared to what is already known on LITAF. Mice are injected with P. gingivalis LPS (less than 0.001 μg/20 g mouse. Blood is drawn at 60 min intervals and cytokine concentrations measured in triplicate. Serial dilutions of serum samples are measured by ELISA for TNF and IL-1, IL-6 and IL-12 (Endogen Inc., Boston USA; Li, et al., Infect Immun, 70:3915-3922, 2002; Li, et al., Circulation, 105:861-867, 2002; Chi, et al., Circulation, 110: 1678-1685, 2004). Absorbance values are read at 450 nm and are converted to concentrations (ng/ml) in the serum by comparison with the respective standard curve of each cytokine. Data are processed by non-parametric statistics and given as median values plus or minus median deviation. More details of these protocols are provided in (Zhou, et al., Infect Immun, 73:935-943, 2005).

3. In vivo testing of angiogenesis. Angiogenesis is tested to validate the role of STAT6(B) and LITAF-STAT6(B) complex in angiogenesis. All animals (STAT6(B)−/−, double LITAF-STAT6(B)−/− and wild-type) (n=6/group) receive sterile injections of 2×500 μl matrigel (Becton Dickinson) sub-cutaneously (dorsally). To promote vessel proliferation, the matrigel plug contains either buffer or LPS (5 ug, 1 ug; 0.1 ug,). OFGF (50 nM) is used as a positive control.

a. Intravital microscopy. Computer models are employed to allow visualization of angiogenesis and vessel morphology in all animals by intravital microscopy (Jain, et al., Nat Rev Cancer, 2:266-276, 2002). An upright microscope (Axioplan; Zeiss) available that is equipped with transillumination, fluorescence epi-illumination and an intensified cooled coupled device camera (C2400-88; Sony) is used. FITC labeled dextran (2×10⁶ molecular weight; 10 mg/ml, 100 μl; Sigma-Aldrich) is injected into the animals' tail veins to contrast and enhance functional blood vessels. Five randomly selected locations of matrigel (500×340 μm² of each) are visualized, examined by intravital fluorescence microscopy and recorded through a frame grabber board (Data Translation Inc.) for image digitization on a computer (IBM Computer Inc.). Vascular parameters such as functional vessel density (the total length of perfused microvessels/unit area) and vessel diameter and number of vessel segments (distinct individual vessel segments connected to each other at the branching point) are analyzed by tracing each vessel segment using NIH Image 1.63 freeware on these captured images and by calculating the data using a macro in Microsoft Excel 2003 as described elsewhere (Jain, et al., Nat Rev Cancer, 2:266-276, 2002). The number of vessel segments is an indicator of vessel branching. Blood vessels are divided into many more segments if there are more branches.

b. Immunohistochemistry: Six days after matrigel placement, the animals are euthanized and the matrigel plug removed, fixed and immersed in paraffin. Immunohistochemistry is performed using a routine protocol. Primary anti-CD31 antibodies (anti-PECAM-1, BD Pharmingen) at 1:300 dilution are used. All histology slide preparation is performed according to standard published protocols (Li, et al., Infect Immun, 70:3915-3922, 2002; Li et al., Circulation, 105:861-867, 2002; Han and Amar, J Biol Chem, 279:2832-2840, 2004). The number of capillaries are counted under the microscope in five different fields in each of the three slices taken from different parts of each plug. The number of capillaries detected in slices from the plugs containing the studied compounds are compared among all groups. Additionally, the number of separately detected endothelial PECAM-positive single cells, not connected with capillaries, migrating to the matrigel are calculated.

c. Image Analysis. Quantification of immunostained cells in each matrigel plug section is accomplished using a computer-assisted image analysis system. Immunostained sections are viewed through a microscope with a color video camera and the images are projected onto a monitor. Image Pro-Plus software is used to aid in counting cells. Cells are counted in sixteen evenly spaced non-overlapping fields (4×4, 500× magnification). The results are expressed as cell number per unit area of tissue cross section (mm²). Cells are counted as described above.

d. Statistical Analysis. ANOVA is used to determine the significance of each of the endpoints measured for all groups tested (saline vs. LPS in LITAF−/−, STAT6(B)−/−, double LITAF-STAT6(B)−/− and wild-type at various time points and doses). For the inflammatory experiments, the relationship between survival rate and inflammatory mediators (cytokine) is established by calculating Pearson correlation coefficients. For the angiogenesis, number of new blood vessels, vessel segment, and immunostained cells are also established by calculating Pearson correlation coefficients. N=6 gives a sufficient sample size to determine statistical significance (defined as p<0.05) with the endpoints-chosen, as shown in similar statistical analysis-performed in several recent publications using numeric data obtained from in vivo experiments (Li, et al., Circulation, 105:861-867, 2002; Chi, et al., Circulation, 110: 1678-1685, 2004; Han and Amar, J Biol Chem, 279:2832-2840, 2004; Ahang, et al., J Immunol, 173:3514-3523, 2004).

Systemic response: Double LITAF-STAT6(B)−/− mice will have a substantially diminished capacity to produce cytokines in the unsensitized animals, resulting in a decrease in LPS-induced lethality. This result is based on the role of TNF and several proinflammatory cytokines as mediators of LPS-induced lethality in the unsensitized as well as the fact that TNF is known to participate in a positive feedback mechanism that can augment the production of LPS-inducible cytokines (e.g., IL-6). Thus, decreased cytokine production in the LITAF-STAT6(B)−/− animals results in a broader reduction in the expression of additional cytokines and, consequently, not only improved resistance to LPS-induced lethality but also to diseases with cytokine dysregulation (i.e., periodontal disease, sepsis, Crohn's). It was expected that, based on positive feedback mechanism, the levels and kinetics of cytokine production cytokines are more pronounced in the LITAF-STAT6(B)−/− than in LITAF−/− animals compared with control animals). Thus, the levels and kinetics of cytokine production are reduced and slowed (respectively) in the knockout mice. Overall, the phenotype of the double LITAF-STAT6(B)−/− mice is similar to that of the TNF receptor-deficient mice (p55−/−) that we have previously studied (Amar, J Inflamm, 47:180-189, 1995). It was expected that the D-Gal sensitized LITAF-STAT6(B)−/− animals are also more resistant to LPS-induced lethality than the wild type mice. This was anticipated since LPS-induced lethality in mediated by TNF. It was also anticipated that the LITAF-STAT6(B)−/− mouse show some important signs of LPS immunity certainly more than what will be observed in the LITAF−/− animals.

Angiogenesis: We expected that STAT6(B)−/− have a substantially diminished capacity to produce neo-vessels while the LITAF-STAT6(B)−/− mice behave like the wild type control animals. This result is based on the role of STAT6(B) in VEGF regulation and angiogenesis in vitro. Overall, we expected that the phenotype of the LITAF-STAT6(B)−/− mice be similar to that of the HO-1 deficient mice (Krampert, et al., J Biol Chem, 280:23844-23852, 2005; Cisowski, Biochem Biophys Res Commun, 326:670-676, 2005). Tests of two variants of B16 melanoma derived from C57BL/6 mice, B16F1 and B16F10 in STAT6(B)−/− animals are used to evaluate the effect on tumor growth and progression.

1. An expression vector comprising a nucleic acid sequence which encodes the protein STAT6(B) (SEQ ID NO: 2), or a biologically active fragment thereof.

2. The expression vector of claim 1 wherein the nucleic acid sequence comprises SEQ ID NO: 4.

3. An isolated protein comprising the amino acid sequence of STAT6(B) (SEQ ID NO: 2), or a biologically active fragment thereof.

4. The isolated protein of claim 3 complexed with the protein LITAF or a biologically active fragment of LITAF thereof.

5. A cell that exogenously expresses the protein STAT6(B) (SEQ ID NO: 2), or a biologically active fragment thereof.

6. The cell of claim 5 that further expresses the protein LITAF, or a biologically active fragment thereof, effective to form a complex with the exogenously expressed STAT6(B).

7. A method for modulating cytokine expression, the method comprising introducing into a cell a composition comprising STAT6(B) (SEQ ID NO: 2), or a biologically active fragment thereof, effective to modulate cytokine expression in the cell.

8. The method of claim 7 wherein the cytokine is selected from the group consisting of IL-1α, IL-10, GRO, VEGF and RANTES.

9. The method of claim 7 further comprising introducing LITAF, or a biologically active fragment thereof, effective to form a complex of the introduced LITAF with the introduced STAT6(B) in the cell.

10. The method of claim 9 wherein the cytokine is selected from the group consisting of TNF-α, IL-1α, IL-10, GRO, RANTES, IFN-γ, VEGF, MCP-1 and MCP-2.

11. A method for modulating cytokine expression, the method comprising introducing into a cell a composition comprising LITAF, or a biologically active fragment thereof, effective to modulate cytokine expression in the cell.

12. The method of claim 11 wherein the cytokine is selected from the group consisting of TNF-α, VEGF and IL-1β.

13. The method of claim 8 wherein said cytokine expression modulation stimulates angiogenesis.

14. The method of claim 8 wherein said cytokine expression modulation stimulates the immune response to an antigen.

15. The method of claim 8 wherein said cytokine expression modulation treats a disease selected from the group consisting of cancer, diabetes and inflammatory diseases.

16. The method of claim 8 wherein said cytokine expression modulation stimulates the processes of tubulogenesis.

17. A null mutant animal comprising a homozygous disruption in the endogenous genes that code for LITAF, wherein the disruption results in the lack of functional LITAF protein expression and wherein the animal shows reduced cytokine production in response to LPS stimulation.

18. The null mutant animal of claim 17, wherein the disruption is selected from a group consisting of insertions, deletions and mutations.

19. The null mutant animal of claim 17, wherein said animal is selected from a group consisting of mice, rats, canines, sheep, cattle, porcine and felines.

20. A cell or cell line derived from the null mutant animal of claim 17.

21. A null mutant animal comprising a homozygous disruption in the endogenous genes that code for STAT6(B), wherein the disruption results in the lack of functional STAT6(B) protein expression and wherein the animal shows reduced cytokine production in response to LPS stimulation.

22. The null mutant animal of claim 21, wherein the disruption is selected from a group consisting of insertions, deletions and mutations.

23. The null mutant animal of claim 21, wherein said animal is selected from a group consisting of mice, rats, canines, sheep, cattle, porcine and felines.

24. A cell or cell line derived from the null mutant animal of claim 21.

25. A null mutant animal comprising a homozygous disruption in the endogenous genes that code for LITAF and STAT6(B), wherein the disruption results in the lack of functional LITAF and STAT6(B) protein expression and wherein the animal shows reduced cytokine production in response to LPS stimulation.

26. The null mutant animal of claim 25, wherein the disruption is selected from a group consisting of insertions, deletions and mutations.

27. The null mutant animal of claim 25, wherein said animal is selected from a group consisting of mice, rats, canines, sheep, cattle, porcine and felines.

28. A cell or cell line derived from the null mutant animal of claim 25.

29. A method for identifying a small molecule characterized by the ability to inhibit p53/LITAF binding, said method comprising:
a. providing a library of small molecules to be screened for the ability to inhibit p53/LITAF binding;
b. forming a reaction mixture comprising a small molecule to be screened for the ability to inhibit p53/LITAF binding, and a mixture of p53 or an active fragment thereof and the LITAF promoter region or an active fragment thereof;
c. incubating the reaction mixture of step b) for a period of time and under conditions appropriate for p53/LITAF binding;
d. determining the extent of p53/LITAF binding following the incubation of step c);
e. comparing the amount p53/LITAF binding determined in step d) to the amount of p53/LITAF binding detected in an otherwise identical incubation mixture which does not include a small molecule to be screened for the ability to inhibit p53/LITAF binding, a decrease in the binding determined in step d) to that of the otherwise identical incubation mixture being indicative of the small molecule of step b) being characterized by the ability to inhibit p53/LITAF promoter region binding.

30. The method of claim 29 wherein said p53-LITAF binding complex is detected by electrophoresis.

31. The method according to claim 29 wherein said p53-LITAF binding complex is detected by electromobility shift assay (EMSA).

32. A method for identifying a small molecule characterized by the ability to inhibit STAT6(B)/LITAF interaction, said method comprising:
a. providing a library of small molecules to be screened for the ability to inhibit STAT6(B)/LITAF interaction;
b. forming a reaction mixture comprising a small molecule to be screened for the ability to inhibit STAT6(B)/LITAF interaction, and a mixture of STAT6(B) or an active fragment thereof and LITAF or an active fragment thereof;
c. incubating the reaction mixture of step b) for a period of time and under conditions appropriate for STAT6(B)/LITAF interaction;
d. determining the extent of STAT6(B)/LITAF interaction following the incubation of step c);
e. comparing the amount STAT6(B)/LITAF interaction determined in step d) to the amount of STAT6(B)/LITAF interaction detected in an otherwise identical incubation mixture which does not include a small molecule to be screened for the ability to inhibit STAT6(B)/LITAF interaction, a decrease in the interaction determined in step d) to that of the otherwise identical incubation mixture being indicative of the small molecule of step b) being characterized by the ability to inhibit STAT6(B)/LITAF interaction.

33. The method of claim 32 wherein said STAT6(B)-LITAF interaction is detected by electrophoresis.

34. The method according to claim 32 wherein said STAT6(B)-LITAF interaction is detected by electromobility shift assay (EMSA).

35. A method of stimulating angiogenesis by introducing a compound identified by the method of claim 32.

36. A method of stimulating angiogenesis comprising administering a compound that blocks the interaction of STAT6(B) with LITAF.

37. A method for identifying a small molecule characterized by the ability to promote STAT6(B)/LITAF interaction, said method comprising:
a. providing a library of small molecules to be screened for the ability to promote STAT6(B)/LITAF interaction;
b. forming a reaction mixture comprising a small molecule to be screened for the ability to promote STAT6(B)/LITAF interaction, and a mixture of STAT6(B) or an active fragment thereof and LITAF or an active fragment thereof;
c. incubating the reaction mixture of step b) for a period of time and under conditions appropriate for STAT6(B)/LITAF interaction;
d. determining the extent of STAT6(B)/LITAF interaction following the incubation of step c);
e. comparing the amount STAT6(B)/LITAF interaction determined in step d) to the amount of STAT6(B)/LITAF interaction detected in an otherwise identical incubation mixture which does not include a small molecule to be screened for the ability to promote STAT6(B)/LITAF interaction, a increase in the interaction determined in step d) to that of the otherwise identical incubation mixture being indicative of the small molecule of step b) being characterized by the ability to promote STAT6(B)/LITAF interaction.

38. The method of claim 37 wherein said STAT6(B)-LITAF interaction is detected by electrophoresis.

39. The method according to claim 37 wherein said STAT6(B)-LITAF interaction is detected by electromobility shift assay (EMSA).

40. A method of inhibiting angiogenesis by introducing a compound identified by the method of claim 37.

41. A method of inhibiting angiogenesis comprising administering a compound that promotes the interaction of STAT6(B) with LITAF.

42. A method of stimulating angiogenesis comprising administering STAT6(B) to a tissue capable of undergoing angiogenesis.

43. The method of claim 42, wherein said STAT6(B) is introduced by transfection of an expression construct comprising a nucleotide sequence encoding STAT6(B) or a functional fragment thereof into one or more cells of said tissue.

44. The method of claim 42, wherein said STAT6(B) is administered by transfection of said protein or functional fragment thereof into said tissue.

45. The method of claim 42, wherein said tissue is located in an organism and said introduction of said STAT6(B) is either local or systemic.

46. A method of inhibiting angiogenesis comprising introducing LITAF to a tissue capable of undergoing angiogenesis.

47. The method of claim 46, wherein said LITAF is introduced by transfection of an expression construct comprising a nucleotide sequence encoding LITAF or a functional fragment thereof into one or more cells of said tissue.

48. The method of claim 46, wherein said LITAF is administered by the transfection of said protein or functional fragment thereof into said tissue.

49. The method of claim 46, wherein said tissue is located in an organism and said introduction of said LITAF is either local or systemic.

50. A method for identifying a small molecule characterized by the ability to inhibit STAT6(B)/VEGF promoter interaction, said method comprising:
a. providing a library of small molecules to be screened for the ability to inhibit STAT6(B)/VEGF promoter interaction;
b. forming a reaction mixture comprising a small molecule to be screened for the ability to inhibit STAT6(B)/VEGF promoter interaction, and a mixture of STAT6(B) or an active fragment thereof and a nucleic acid encoding a VEGF promoter or an active fragment thereof;
c. incubating the reaction mixture of step b) for a period of time and under conditions appropriate for STAT6(B)/VEGF promoter interaction;
d. determining the extent of STAT6(B)/VEGF promoter interaction following the incubation of step c);
e. comparing the amount STAT6(B)/VEGF promoter interaction determined in step d) to the amount of STAT6(B)/VEGF promoter interaction detected in an otherwise identical incubation mixture which does not include a small molecule to be screened for the ability to inhibit STAT6(B)/VEGF promoter interaction, a increase in the interaction determined in step d) to that of the otherwise identical incubation mixture being indicative of the small molecule of step b) being characterized by the ability to promote STAT6(B)/VEGF promoter interaction.

51. The method of claim 50 wherein said STAT6(B)-VEGF interaction is detected by a method selected from a group consisting of electrophoresis, electromobility shift assay (EMSA) and Western blot.
